(19) United States
(12) Patent Application Publication
(10) Pub. No.: US 2026/0207459 A1
(43) Pub. Date: Jul. 23, 2026

(54) SUGARCANE DERIVED SEMI-SYNTHETIC COSMETIC INGREDIENTS, METHODS OF PRODUCTION THEREOF, AND PERSONAL CARE PRODUCTS DERIVED THEREFROM

(71) Applicant: AMYRIS, INC., Emeryville, CA (US)

(72) Inventors: Greg Oniel BUCHANAN, Emeryville, CA (US); Anushree DAS, Emeryville, CA (US); Lovedeep KAUR, Emeryville, CA (US); Mikhail MOTORNOV, Emeryville, CA (US); Daan Thorn LEESON, Emeryville, CA (US); Punitha VEDANTHAM, Emeryville, CA (US)

(21) Appl. No.: 19/157,872

(22) PCT Filed: Mar. 1, 2024
(86) PCT No.: PCT/US2024/018025

§ 371 Date: Aug. 19, 2025

Related U.S. Application Data
(60) Provisional application No. 63/449,411, filed on Mar. 2, 2023.

(51) Int. Cl. A61K 8/49; A61K 8/31; A61K 8/34; A61K 8/37; A61Q 19/08; C07C 67/08; C07C 69/003; C07D 307/62; C12P 5/00

(52) U.S. Cl. CPC A61K 8/4973; A61K 8/31; A61K 8/347; A61K 8/37; A61Q 19/08; C07C 67/08; C07C 69/003; C07D 307/62; C12P 5/007

(57) ABSTRACT

The present invention provides compounds derived from farnesene and methods of producing the compounds from farnesene. In addition, compositions and methods of using the compositions as personal care products and other consumer products are provided, wherein the compositions comprise compounds derived from farnesene.

### CROSS-REFERENCE TO RELATED APPLICATIONS

This application claims priority to, and the benefit, of 63/449,411 filed Mar. 2, 2023, the entirety of which is hereby incorporated by reference for all purposes.

### FIELD

The present invention relates to sustainable, sugarcane-derived, semi-synthetic personal care product ingredients, their method of production, and personal care products derived solely from sugarcane.

### BACKGROUND

Many personal care products, such as cosmetics, contain chemical materials that are derived from petroleum or extracted from plants. However, the use of these materials depletes natural resources, is not sustainable, and, petroleum derived chemical materials are not ideal in personal care product formulations. Petroleum derived oils such as mineral oil, when applied to the skin, are occlusive in nature. They can seal off the skin from air and water, which can block pores and the skin's natural respiration process. Blocked pores can result in trapped dirt and oil, leading to blackheads, pimples, and other undesirable skin conditions. The occlusive nature of petroleum derived oils can also create a warm, moist environment for bacteria or fungi to grow. Other ingredients for cosmetics are continually evaluated and can be found to be toxic. For example, certain siloxanes, typically added to give products a slippery, smooth, and silky effect for easy application, were recently banned in the European Union, and cannot be used in rinse-off cosmetics or leave-in cosmetics at concentrations greater than 0.1%. Bio-renewably sourced alternatives to petroleum-derived products, plant-extracted products, and toxic siloxanes that can give products high sheen, shine, and/or gloss would be highly advantageous.

There is also a continuing need to incorporate these sustainable, bio-renewable sources into common ingredients in cosmetic personal care products, such as vitamin C and vitamin C esters. Less collagen is produced as aging occurs, but vitamin C and vitamin C esters help to promote collagen production to create a tighter look in the face and brighten the skin's appearance. However, conventional vitamin C possesses a notable vulnerability to rapid degradation when exposed to air, light, and heat (Yuan, J.-P.; Chen, F. J. Agric. Food Chem. 1998, 46 (12), 5078-5082).

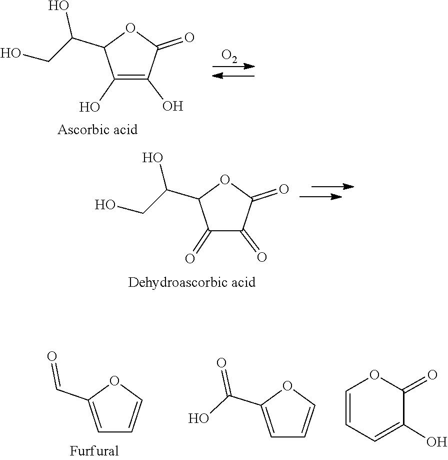

This inherent instability poses a formidable obstacle for formulation into skincare preparations since its efficacy can rapidly diminish due to degradation, and the side products generated might be carcinogenic (National Toxicology Program. NTP Toxicology and Carcinogenesis Studies of Furfural (CAS No. 98-01-1) in F344/N Rats and B6C3F1 Mice (Gavage Studies). Natl Toxicol Program Tech Rep Ser. 1990 March; 382:1-201. PMID: 12692654).

Vitamin C esters are alternatives to vitamin C that are fat soluble. The skin absorbs and utilizes vitamin C ester more readily than vitamin C, which is water-soluble and poorly penetrates the epidermis stratum corneum barrier. Vitamin C esters typically have a long shelf-life, and compared to vitamin C, are less prone to oxidation and are gentler on the skin. One of the potential benefits of vitamin C ester is improved skin elasticity with regular use. This is achieved through the stimulation of collagen fibers within the skin, which are synthesized less with aging. Lipophilic vitamin C esters improve skin diffusion and delivery to promote collagen production. Stronger, more plentiful protein fibers create a tighter look in the face and diminish the appearance of wrinkles. Vitamin C ester is also believed to help brighten the skin's complexion, reduce the appearance of hyperpigmentation and dark spots, and improve its overall texture and tone. A common commercial form of a vitamin C ester is tetrahexadecyl ascorbate (THDA), however THDA is costly, and skincare brands frequently incorporate only small amounts of THDA in their formulations. Given the beneficial use of vitamin C ester derivatives in cosmetic formulations, it would be valuable to develop additional vitamin C ester derivatives that exhibit high degrees of dermis penetration so that less of the Vitamin C ester derivative is required and/or are made from bio-renewable sources in cost-efficient and sustainable synthetic procedures.

There is also a continuing need for new feedstock materials, preferably derived from renewable sources. In particular, many commercially available consumer and industrial products contain solvents that include volatile organic compounds (VOCs), which are environmentally unfriendly. Solvent compositions are useful in a wide range of products for removing organic and other substances from furniture, floors, walls, mechanical devices, automobiles, bicycles, clothing, skin, and the like. Useful solvents have advantageous properties, such as vapor pressures, viscosities, degreasing powers, stabilities, odor and/or color. For many applications, advantageous safety profiles are desired. Solvent compositions with low environmental impact and low VOC content are needed, and few solvent compositions are currently made from sustainable, renewable sources.

Therefore, there is a need for sustainable and renewably-sourced intermediates and compounds for personal care products that are advantageous as active compounds (for example, are capable of penetrating deeper into the skin) and/or as additives to improve and facilitate easy application and active ingredient delivery (by improving the spreadability, shininess, gloss, and/or viscosity of the product). There is also a continuing need for new solvent compositions with desired properties that are made from sustainable, renewable sources. Embodiments described herein meet these and other needs.

### SUMMARY

In one aspect, provided herein are isolated or purified bio-renewably sourced compounds. In one embodiment, the bio-renewably sourced compound is a compound of Formula (II):

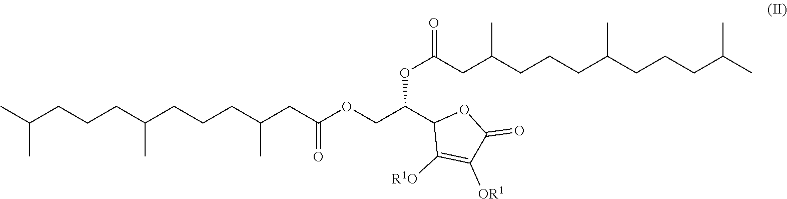

wherein R¹ is independently, at each occurrence, selected from H and

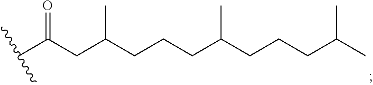 [SMILES: *C(=O)CC(C)CCCC(C)CCCC(C)C]

or a salt and/or stereoisomer thereof.

Vitamin C, also known as ascorbic acid, is a potent antioxidant that helps protect cells from damage caused by free radicals. It is also involved in the production of collagen, a protein that helps to give structure and elasticity to the skin, joints, and blood vessels, and for this reason, has been frequently featured in skincare formulations. Despite its ability to promote collagen production, it is susceptible to rapid degradation into carcinogenic side products. One popular alternative to vitamin C are vitamin C esters, fat soluble forms of vitamin C, that have a longer shelf life, are less susceptible to oxidation, are less acidic, and gentler on the skin than vitamin C. However, vitamin C esters, for example the commonly used tetrahexadecyl ascorbate (THDA) can be expensive; due to the price, skincare formulations frequently only incorporate a small amount. Therefore, it would be highly advantageous to develop vitamin C esters that have a high degree of dermis penetration, which would require less compound in the formulation, and/or are made by a cost-efficient synthesis using a bio-renewable intermediate. As described herein in Example 12, certain compounds of Formula (II) exhibited better penetration to the dermis than THDA. The dermis is where collagen is found, suggesting that these compounds will promote better collagen production, and therefore, diminish the appearance of aging and create a tighter look, than THDA.

In one embodiment, the compound of Formula (II) is di(hexahydrofamesyl) ascorbate (Compound (II-A)):

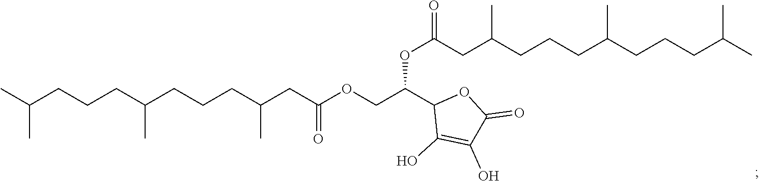 [SMILES: CC(C)CCCC(C)CCCC(C)CC(=O)OC[C@H](OC(=O)CC(C)CCCC(C)CCCC(C)C)C1OC(=O)C(O)=C1O]

or a salt and/or stereoisomer thereof.

In one embodiment, the compound of Formula (II) is tri(hexahydrofarnesyl) ascorbate (Compound (II-B)):

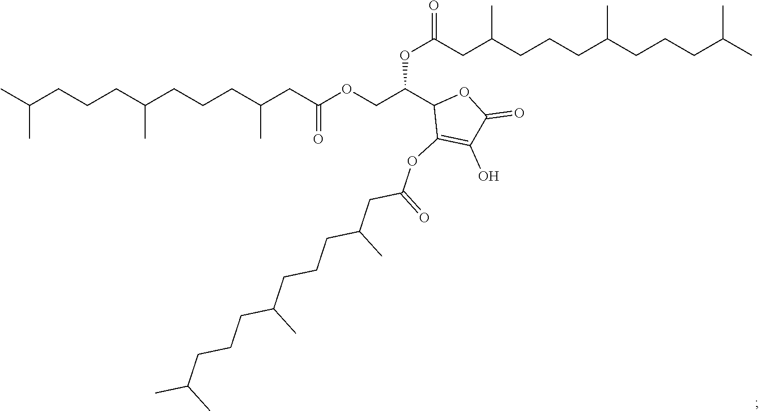 [SMILES: CC(C)CCCC(C)CCCC(C)CC(=O)OC[C@H](OC(=O)CC(C)CCCC(C)CCCC(C)C)C1OC(=O)C(O)=C1OC(=O)CC(C)CCCC(C)CCCC(C)C]

or a salt and/or stereoisomer thereof.

In one embodiment, the compound of Formula (II) is tetra(hexahydrofarnesyl) ascorbate (Compound (II-C)):

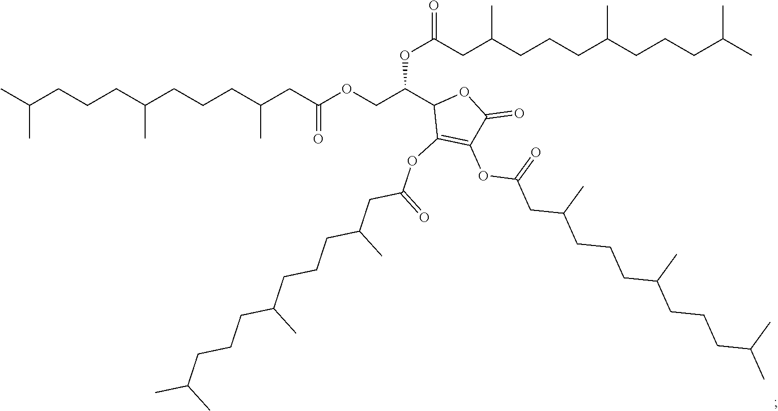 [SMILES: CC(C)CCCC(C)CCCC(C)CC(=O)OC[C@H](OC(=O)CC(C)CCCC(C)CCCC(C)C)C1OC(=O)C(OC(=O)CC(C)CCCC(C)CCCC(C)C)=C1OC(=O)CC(C)CCCC(C)CCCC(C)C]

or a salt and/or stereoisomer thereof.

In another embodiment, the bio-renewably sourced compound is 3,7,11-trimethyldodecyl 3,7,11-trimethyldodecanoate (Compound (I-C)):

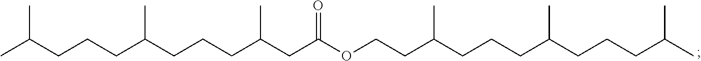 [SMILES: CC(C)CCCC(C)CCCC(C)CCOC(=O)CC(C)CCCC(C)CCCC(C)C]

or a salt and/or stereoisomer thereof.

In another aspect, provided herein are processes for producing, separating, and purifying a compound of Formula (II) or Compound (II-A), (II-B), (II-C), or (I-C).

Bio-renewably sourced compounds and intermediates that can act as additives to personal care products to help increase the shine, spreadability, gloss, and/or viscosity of the product are also described herein. High shine, spreadability, gloss, and/or viscosity facilitate easy application and/or the delivery of active ingredients. As discussed in Example 11, compounds that easily spread and are slippery often have high foam and lathering ability, which is important for face washes and soap. Compounds that spread easily and smoothly also feel soft on the skin, a preferred property for lotion and hand creams. Lastly, slippery additives can help to detangle hair.

Therefore, also provided herein are processes for producing, separating, and purifying bio-renewably sourced compounds and intermediates. Also provided herein are personal care products comprising these compounds.

In one embodiment, provided herein, is a process for producing, separating, and purifying a compound of Formula (I):

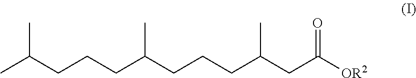

or a salt and/or stereoisomer thereof wherein R² is selected from a straight or branched C₁₋₆alkyl.

In one embodiment, the compound of Formula (I) is ethyl-hexahydrofamesoate (Compound (I-A)):

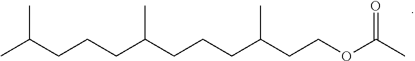 [SMILES: CC(=O)OCCC(C)CCCC(C)CCCC(C)C]

In another embodiment, the compound of Formula (I) is isopropyl-hexahydrofarnesoate (Compound (I-B)):

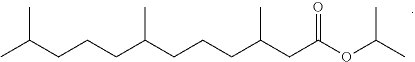 [SMILES: CC(C)CCCC(C)CCCC(C)CC(=O)OC(C)C]

Many bio-renewable farnesene derivatives, including those described herein, are made from hexahydrofarnesoic acid (HHFA):

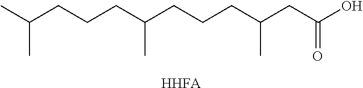

Therefore, also provided herein is a solvent-free sustainable and scalable process that uses a non-toxic and recyclable catalyst in combination with aqueous-based reagents for producing, separating, and purifying HHFA or a salt and/or stereoisomer thereof.

HHFA is generated in a single step by the oxidation of the corresponding alcohol hexahydrofarnesol (HHF):

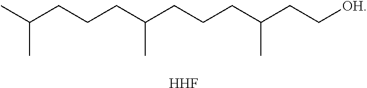

Commonly used reagents for the conversion of alcohols to acids are heavy metal-based oxidants such as hexavalent chromium salts, manganese oxides, or lead salts. These are toxic species, which creates serious handling and disposal problems. Such reagents also require stoichiometric amounts of oxidants that must be freshly prepared and generate side products, rendering them undesirable for large-scale synthesis. For example, Jones reagent (a chromium-based reagent) must be freshly prepared by combining chromium trioxide and sulfuric acid and the disposal of chromium waste requires a standalone metal waste stream. The synthesis described herein is highly advantageous in that it is solvent-free and uses a non-metal-based oxidant, TEMPO, and aqueous-based reagents, bleach with sodium chlorite. As described in Example 13, the sustainable and solvent-free process was conducted on a 300 g scale and afforded HHFA in 94% yield with a purity of 92%.

Also described herein are personal care products comprising a compound of Formula (I) or (II) or Compound (I-A), (I-B), (I-C), (II-A), (II-B), or (II-C), alone or in combination with one or more additional components selected from an emollient, a sensory modifier, an organic solvent, a co-emulsifier, a thickener, a moisturizing agent, an anti-inflammatory agent, a preservative, an anti-aging active, an anti-oxidant, water, and a fragrance.

Also described herein are personal care products comprising a compound of Formula (I) or (II) or Compound (I-A), (I-B), (I-C), (II-A), (II-B), or (II-C), alone or in combination with one or more additional components selected from an emollient, a sensory modifier, an organic solvent, a co-emulsifier, a thickener, a moisturizing agent, an anti-inflammatory agent, a preservative, an anti-aging active, an anti-oxidant, water, and a fragrance, wherein all of the components are derived from sugar cane.

### BRIEF DESCRIPTION OF THE FIGURES

FIG. 1 is a schematic showing the steps from beta-farnesene (which is produced by fermentation from sugarcane) to hexahydrofarnesoic acid.

FIG. 2A is a schematic showing the formation of di/tri/tetra HH-farnesyl ascorbate from hexahydrofarnesoic acid.

FIG. 2B is a schematic showing the formation of HH-farnesyl HH-farnesoate, HH-farnesyl ethanoate, and HH-farnesyl isopropanoate from hexahydrofarnesoic acid.

FIG. 2C is a schematic showing the formation of HH-farnesyl polyglycerol esters from hexahydrofarnesoic acid.

FIG. 3 is a graph showing the simulated distillation of crude HHFA to determine the boiling points of the main components at 1 atmosphere. This was used to predict boiling point targets for compounds of interest at a given vacuum pressure.

FIG. 4 is a graph showing the surface tension values of LEX FEEL, SPI-2005, HDD (distilled), HDD (non-redistilled), PROTOL, RUDOL, ERVOL, and squalane.

FIG. 5A is a graph of the amount of Tetrahexydecyl (tetrahexydecyl ascorbate), Di (di(hexahydrofarnesyl) ascorbate (Compound (II-A))), Tri (tri(hexahydrofarnesyl) ascorbate (Compound (11-B))), and Tetra (tetra(hexahydrofarnesyl) ascorbate (Compound (I1-C))) that passed through the skin into the reservoir at each of the time points (4, 8, and 24 hours) following application to cadaver skin as described in Example 12. The results are also shown in Table 10.

FIG. 5B is a graph of the amount of Tetrahexydecyl (tetrahexydecyl ascorbate), Di (di(hexahydrofarnesyl) ascorbate (Compound (II-A))), Tri (tri(hexahydrofarnesyl) ascorbate (Compound (II-B))), and Tetra (tetra(hexahydrofarnesyl) ascorbate (Compound (II-C))) that accumulated in the dermis and epidermis at the 24 hour time point following application to cadaver skin as described in Example 12. The results are also shown in Table 10.

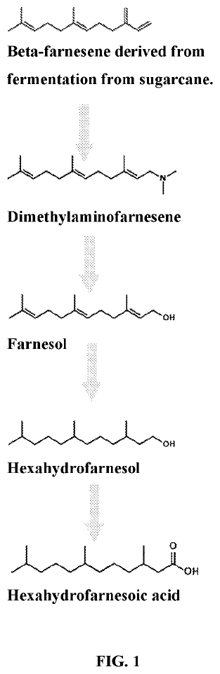

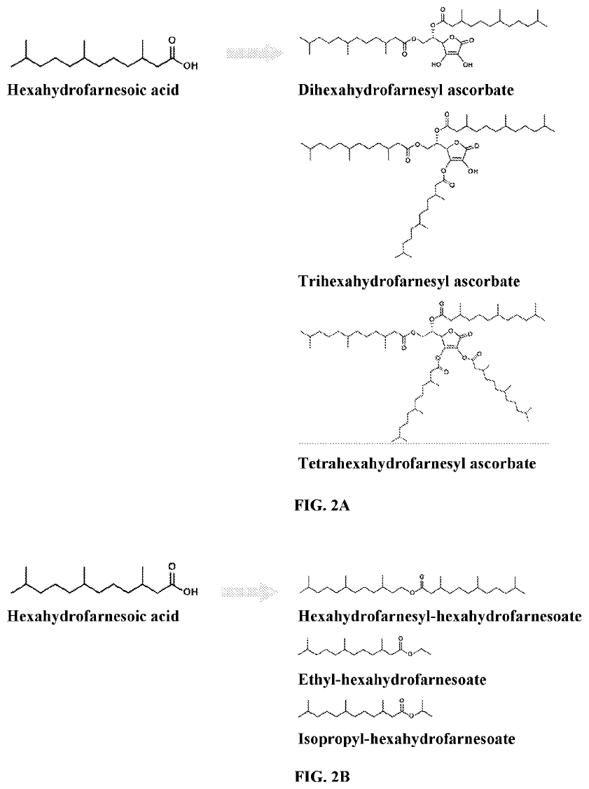

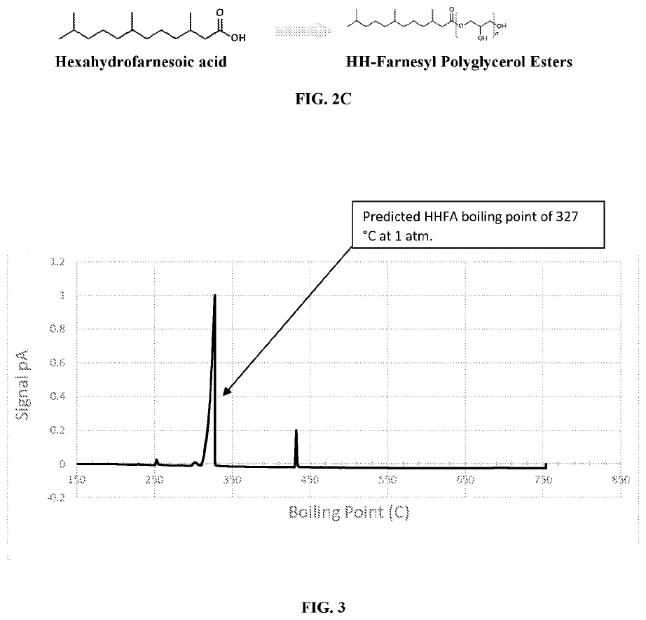

### DETAILED DESCRIPTION OF EMBODIMENTS

### Definitions

As used herein, the terms “beta-farnesene” and “jp-farnesene” refer to a compound having the following structure:

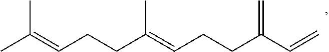

or a stereoisomer thereof. In some variations, β-farnesene comprises a substantially pure stereoisomer of β-farnesene. In other variations, β-farnesene comprises a mixture of stereoisomers, such as cis-trans isomers. In further embodiments, the amount of each of the stereoisomers in the β-farnesene mixture is independently from about 0.1 wt. % to about 99.9 wt. %, from about 0.5 wt. % to about 99.5 wt. %, from about 1 wt. % to about 99 wt. %, from about 5 wt. % to about 95 wt. %, from about 10 wt. % to about 90 wt. %, from about 20 wt. % to about 80 wt. %, based on the total weight of the β-farnesene mixture.

As used herein, the term “bio-based farnesene” refer to farnesene which is biologically produced from microorganisms, in particular, genetically modified microorganisms, by fermentation of renewable carbon sources such as sugar.

As use herein, the term “farnesene derivative” refers to a compound that is chemically derived from farnesene. In preferred embodiments, the farnesene derivative is derived from bio-based farnesene. Non-limiting illustrative examples of farnesene derivatives include dihexahydrofarnesyl ascorbate (Compound (II-A)), trihexahydrofarnesyl ascorbate (Compound (II-B)), tetrahydrofarnesyl ascorbate (Compound (II-C)), hexahydrofarnesyl-hexahydrofarnesoate (Compound (I-C)), ethyl-hexahydrofarnesoate (Compound (I-A)), isopropyl-hexahydrofarnesoate (Compound (I-B)), and hexahydrofarnesene dimer.

As used herein, the term “dihexahydrofarnesyl ascorbate” refers to a compound having the following structure:

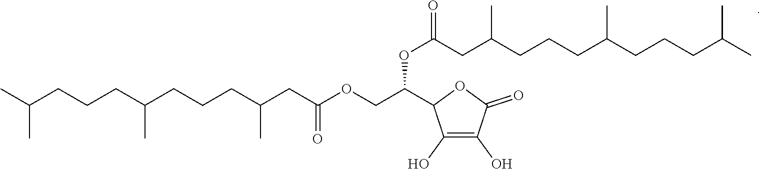 [SMILES: CC(C)CCCC(C)CCCC(C)CC(=O)OC[C@H](OC(=O)CC(C)CCCC(C)CCCC(C)C)C1OC(=O)C(O)=C1O]

As used herein, the term “trihexahydrofarnesyl ascorbate” refers to a compound having the following structure:

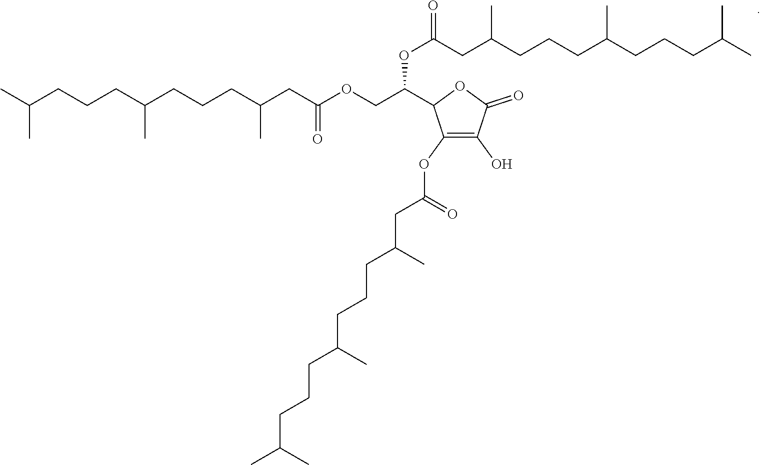 [SMILES: CC(C)CCCC(C)CCCC(C)CC(=O)OC[C@H](OC(=O)CC(C)CCCC(C)CCCC(C)C)C1OC(=O)C(O)=C1OC(=O)CC(C)CCCC(C)CCCC(C)C]

As used herein, the term “tetrahexahydrofarnesyl ascorbate” refers to a compound having the following structure:

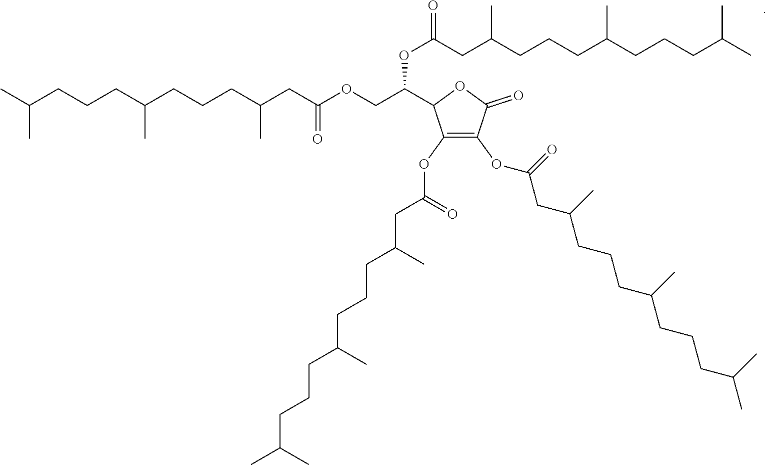 [SMILES: CC(C)CCCC(C)CCCC(C)CC(=O)OC[C@H](OC(=O)CC(C)CCCC(C)CCCC(C)C)C1OC(=O)C(OC(=O)CC(C)CCCC(C)CCCC(C)C)=C1OC(=O)CC(C)CCCC(C)CCCC(C)C]

As used herein, the term “HH-farnesyl polyglycerol esters” refer to a family of compounds having the following structure:

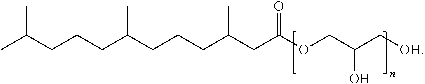

As used herein, the terms “hexahydrofarnesyl-hexahydrofarnesoate” and “HH-farnesyl HH-farnesoate” refer to a compound having the following structure:

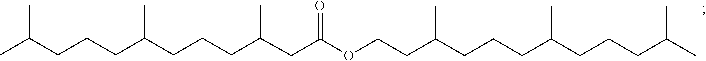 [SMILES: CC(C)CCCC(C)CCCC(C)CCOC(=O)CC(C)CCCC(C)CCCC(C)C]

As used herein, the terms “ethyl-hexahydrofarnesoate” and “HH-farnesyl ethanoate” refer to a compound having the following structure:

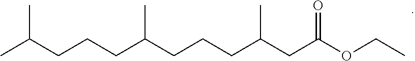 [SMILES: CCOC(=O)CC(C)CCCC(C)CCCC(C)C]

As used herein, the terms “isopropyl-hexahydrofarnesoate” and “HH-farnesyl isopropanoate” refer to a compound having the following structure:

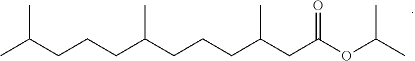 [SMILES: CC(C)CCCC(C)CCCC(C)CC(=O)OC(C)C]

As used herein, the terms “hexahydrofarnesene dimer,” “hydrogenated difarnesene dimer,” and “HDD” refer to compounds having the following structures:

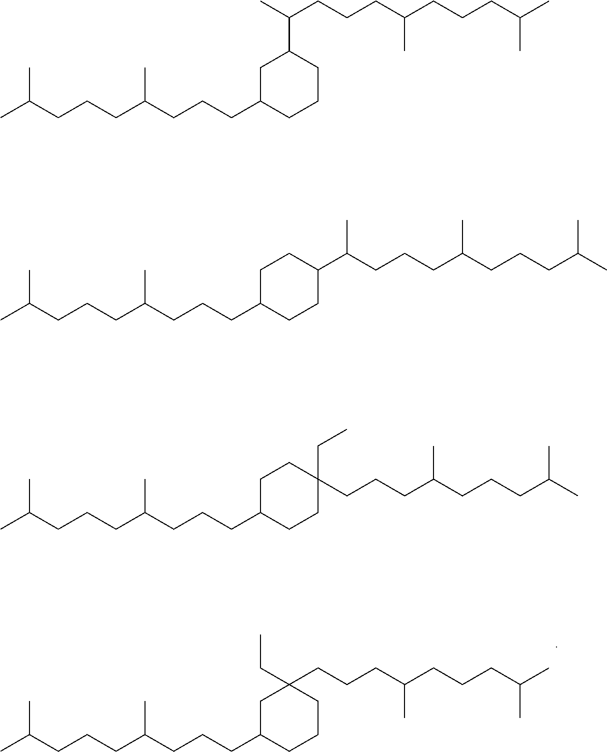

As used herein, the term “squalane” refers to a compound having the following structure:

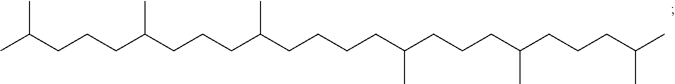 [SMILES: CC(C)CCCC(C)CCCC(C)CCCCC(C)CCCC(C)CCCC(C)C]

As used herein, the term “hemisqualane” and “farnesane” refers to a compound having the following structure:

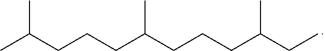

As used herein, the term “dimethylaminofamesene” refers to a compound having the following structure:

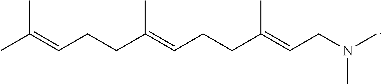 [SMILES: CC(C)=CCC/C(C)=C/CC/C(C)=C/CN(C)C]

As used herein, the term “HH-famesyl dimethylamine oxide” refers to a compound having the following structure:

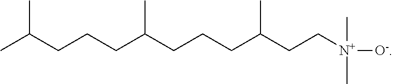

As used herein, the term “farnesol” refers to a compound having the following structure:

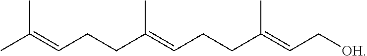 [SMILES: CC(C)=CCC/C(C)=C/CC/C(C)=C/CO]

As used herein, the term “hexahydrofarnesol” or “HHF” refers to a compound having the following structure:

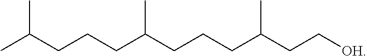 [SMILES: CC(C)CCCC(C)CCCC(C)CCO]

As used herein, the term “hexahydrofarnesoic acid” or “HHFA” refers to a compound having the following structure:

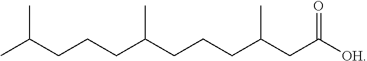 [SMILES: CC(C)CCCC(C)CCCC(C)CC(=O)O]

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₁₋₁₁, C₁₋₁₂, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl groups can be substituted or unsubstituted.

A “salt” of a compound means a salt that is pharmaceutically and/or cosmetically acceptable and possesses the desired activity of the parent compound. “Cosmetically acceptable” salts are any salts that are cosmetically tolerated if used appropriately for a cosmetic treatment when applied to humans and/or mammals. “Pharmaceutically acceptable” salts are those that are generally regarded as safe and non-toxic. It is understood that the pharmaceutically and cosmetically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference, or S. M. Berge et al., “Pharmaceutical Salts,” J. Pharm. Sci. 1977; 66, 1-19 which is also incorporated herein by reference. It is also understood that the compound can have one or more pharmaceutically acceptable and/or cosmetically salts associated with it.

Examples of acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2 hydroxyethanesulfonic acid, benzenesulfonic acid, 4-chlorobenzenesulfonic acid, 2 naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, salicylic acid, and the like.

Examples of acceptable base addition salts include those formed when an acidic proton present in the parent compound is replaced by a metal ion, such as sodium, potassium, lithium, ammonium, calcium, magnesium, iron, zinc, copper, manganese, and aluminum, as salts, and the like. Preferable salts are the ammonium, potassium, sodium, calcium, and magnesium salts. Salts derived from organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

As used herein, the term “about” will be understood by persons of ordinary skill in the art and will vary to some extent on the context in which is used. If there are uses of the term which are not clear to persons of ordinary skill in the art given the context in which is used, ‘about’ may mean up to plus or minus 20% of the particular term.

“Pure” or “purified” as described herein, refers to the purity of a given compound. For example, a compound is “purified” when the given compound is a major component of the composition, i.e., at least 50% w/w pure. Thus, “purified” embraces at least 50% w/w purity, at least 60% w/w purity, at least 70% purity, at least 80% purity, at least 85% purity, at least 90% purity, at least 92% purity, at least 94% purity, at least 96% purity, at least 97% purity, at least 98% purity, at least 99% purity, at least 99.5% purity, and at least 99.9% purity, wherein “substantially pure” embraces at least 90% purity, at least 95% purity, at least 96% purity, at least 97% purity, at least 98% purity, at least 99% purity, at least 99.5% purity, and at least 99.9% purity.

### Isolated or Purified Bio-Renewably Sourced Compounds

In one aspect, provided herein are isolated or purified bio-renewably sourced compounds. In one embodiment, the bio-renewably sourced compound is a compound of Formula (II):

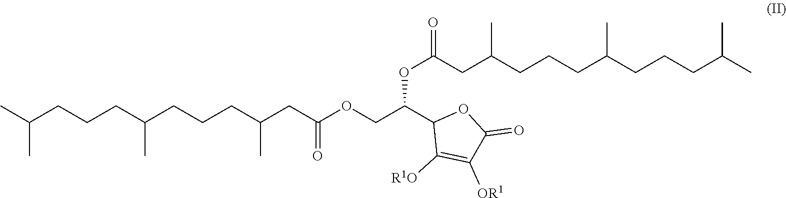

wherein R¹ is independently, at each occurrence, selected from H and

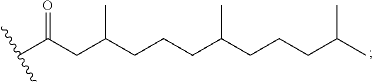 [SMILES: *C(=O)CC(C)CCCC(C)CCCC(C)C]

or a salt and/or stereoisomer thereof.

In one embodiment, the compound of Formula (II) is di(hexahydrofarnesyl) ascorbate (Compound (II-A)):

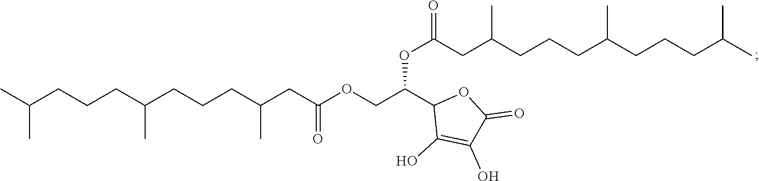 [SMILES: CC(C)CCCC(C)CCCC(C)CC(=O)OC[C@H](OC(=O)CC(C)CCCC(C)CCCC(C)C)C1OC(=O)C(O)=C1O]

or a salt and/or stereoisomer thereof.

In one embodiment, the compound of Formula (II) is tri(hexahydrofarnesyl) ascorbate (Compound (II-B)):

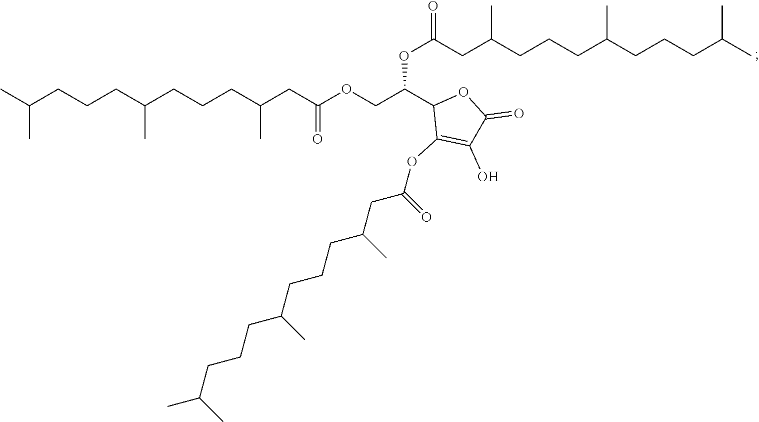 [SMILES: CC(C)CCCC(C)CCCC(C)CC(=O)OC[C@H](OC(=O)CC(C)CCCC(C)CCCC(C)C)C1OC(=O)C(O)=C1OC(=O)CC(C)CCCC(C)CCCC(C)C]

or a salt and/or stereoisomer thereof.

In one embodiment, the compound of Formula (II) is tetra(hexahydrofarnesyl) ascorbate (Compound (II-C)):

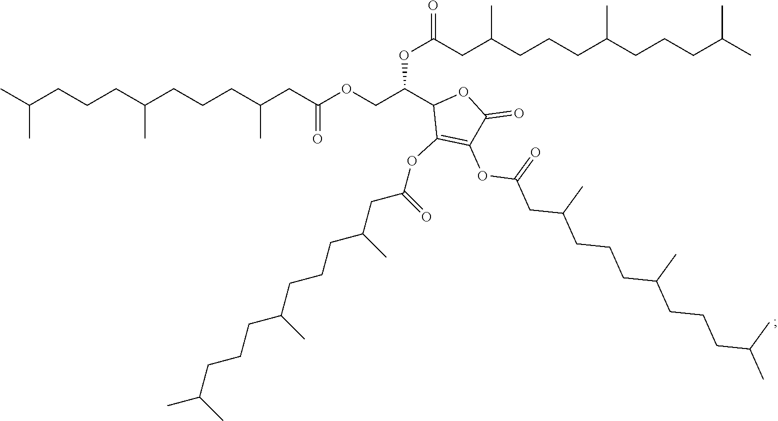 [SMILES: CC(C)CCCC(C)CCCC(C)CC(=O)OC[C@H](OC(=O)CC(C)CCCC(C)CCCC(C)C)C1OC(=O)C(OC(=O)CC(C)CCCC(C)CCCC(C)C)=C1OC(=O)CC(C)CCCC(C)CCCC(C)C]

or a salt and/or stereoisomer thereof.

In one embodiment, the compound of Formula (II) is greater than 90% free of the opposite (R)-enantiomer. In one embodiment, the compound of Formula (II) is greater than 95% free of the opposite (R)-enantiomer. In one embodiment, the compound of Formula (II) is greater than 96%, 97%, 98%, 99%, or 100% free of the opposite (R)-enantiomer.

In an alternative embodiment, the compound of Formula (II) is

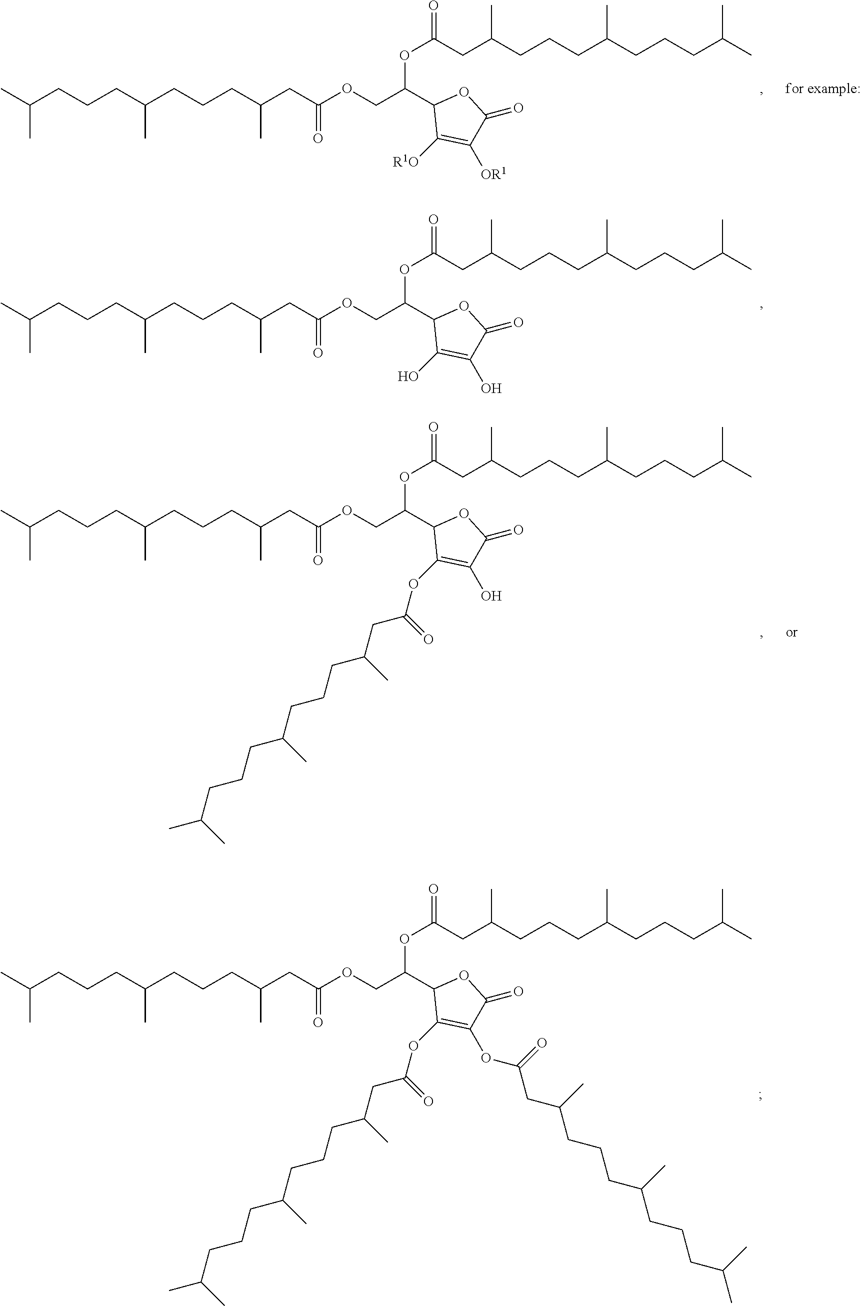

or a salt and/or stereoisomer thereof.

In an alternative embodiment, the compound of Formula (II) is

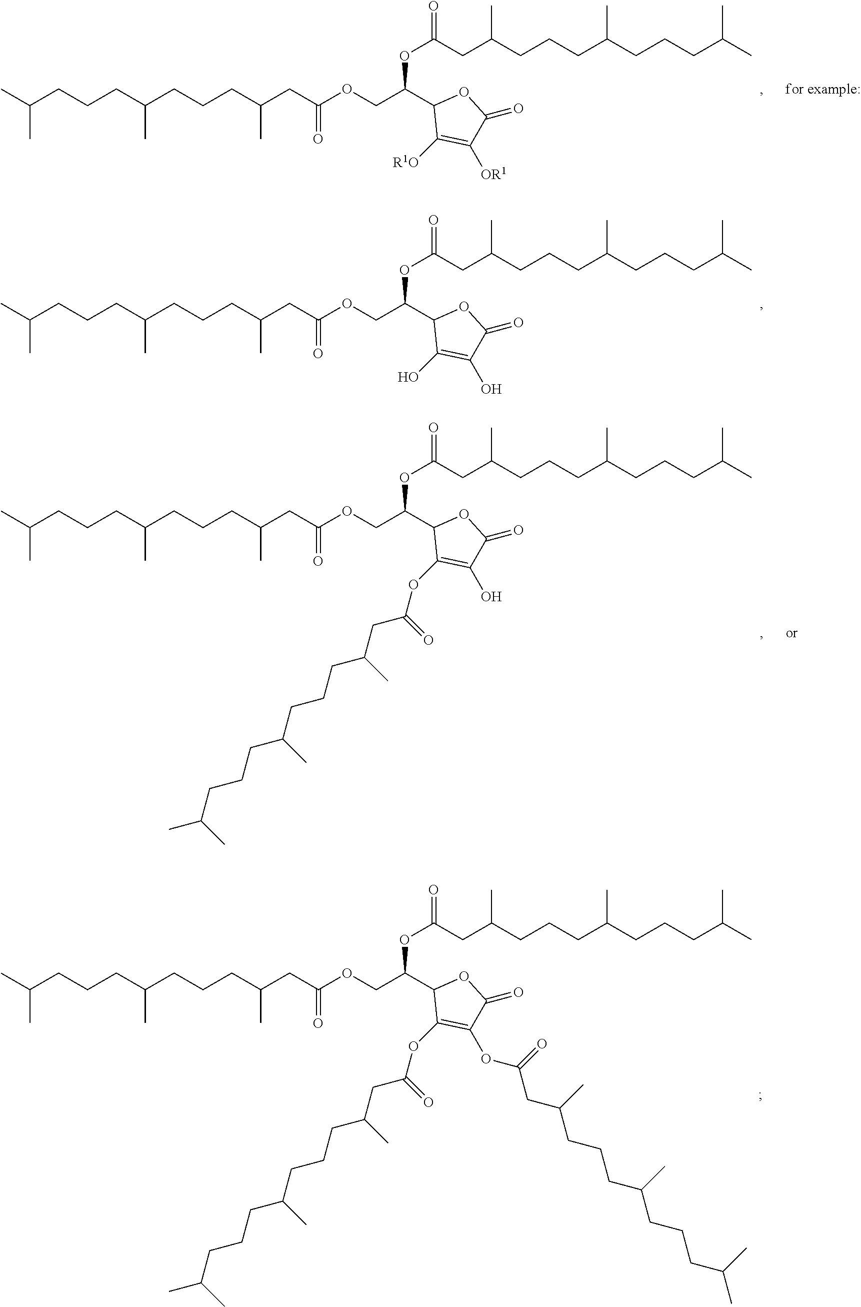

or a salt and/or stereoisomer thereof.

In another embodiment, the bio-renewably sourced compound is 3,7,11-trimethyldodecyl 3,7,11-trimethyldodecanoate (Compound (I-C)):

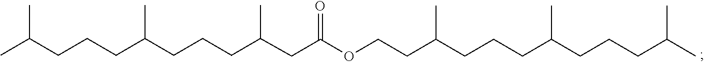 [SMILES: CC(C)CCCC(C)CCCC(C)CCOC(=O)CC(C)CCCC(C)CCCC(C)C]

or a salt and/or stereoisomer thereof.

### Methods of Producing Bio-Renewably Sourced Compounds and Intermediates Thereof

In one aspect, methods are provided to produce a compound of Formula (II):

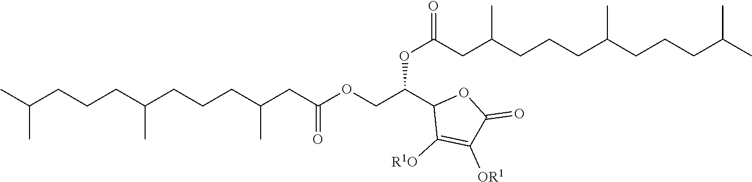

wherein R¹ is independently, at each occurrence, selected from H and

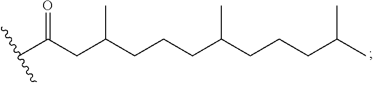 [SMILES: *C(=O)CC(C)CCCC(C)CCCC(C)C]

or a salt and/or stereoisomer thereof.

In one embodiment, the method to produce a compound of Formula (II) comprises:
contacting hexahydrofarnesoic acid:

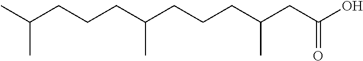 [SMILES: CC(C)CCCC(C)CCCC(C)CC(=O)O]

with ascorbic acid and a coupling agent.

In one embodiment, the ascorbic acid is L-ascorbic acid.

In one embodiment, including any of the foregoing, the coupling agent is selected from N-(3-dimethylaminopropyl)-N′-ethylcarbodiimide hydrochloride (EDC), N,N′-diisopropylcarbodiimide (DIC), and N,N′-dicyclohexylcarbodiimide (DCC). In other embodiments, including any of the foregoing, the coupling agent is selected from hexafluorophosphate azabenzotriazole tetramethyl uranium (HATU), hexafluorophosphate benzotriazole tetramethyl uranium (HBTU), propanephosphonic acid anhydride (T3P), benzotriazole-1-yl-oxy-tris-(dimethylamino)-phosphonium hexafluorophosphate (BOP), benzotriazole-1-yloxytripyrrolidinophosphonium hexafluorophosphate (PyBOP), and 1,1′-carbonyldiimidazole (CDI). In one embodiment, including any of the foregoing, the coupling agent is N-(3-dimethylaminopropyl)-N′-ethylcarbodiimide hydrochloride. In one embodiment, including any of the foregoing, the coupling agent is N,N′-diisopropylcarbodiimide. In one embodiment, including any of the foregoing, the coupling agent is N,N′-dicyclohexylcarbodiimide. In one embodiment, including any of the foregoing, the coupling agent is hexafluorophosphate azabenzotriazole tetramethyl uranium.

In one embodiment, including any of the foregoing, the method further comprises a catalyst, for example 4-dimethylaminopyridine or 1-hydroxybenzotriazole (HOBT).

In one embodiment, including any of the foregoing, the method further comprises the use of a base, for example, pyridine or a base of the formula NR₃ wherein R is selected independently in each instance from H and alkyl wherein NR₃ typically has at least one, and more often, two or three, non-hydrogen R groups. In one embodiment, including any of the foregoing, the base is DIPEA (N,N-diisopropylethylamine) or NEt₃ (triethylamine).

In one embodiment, including any of the foregoing, the method is conducted in a polar aprotic solvent, for example dimethylformamide (DMF), dichloromethane (DCM), tetrahydrofuran (THF), 2-methyltetrahydrofuran (2-MeTHF), ethyl acetate (EtOAc), acetonitrile (MeCN), dimethyl sulfoxide (DMSO), acetone, or N-methylpyrrolidone. In one embodiment, including any of the foregoing, the method is conducted in in N-methylpyrrolidone. In one embodiment, including any of the foregoing, the method is conducted in in DCM. In one embodiment, including any of the foregoing, the method is conducted in in EtOAc. In one embodiment, including any of the foregoing, the method is conducted in in a mixture of solvents, for example, N-methylpyrrolidone and DCM.

In one embodiment, the method produces Compound (II-A) and comprises contacting hexahydrofarnesoic acid with ascorbic acid, 4-dimethylaminopyridine, N-methyl-2-pyrrolidone, and a coupling agent. In one embodiment of the method to produce Compound (II-A), the coupling agent is selected from N-(3-dimethylaminopropyl)-N′-ethylcarbodiimide hydrochloride, N,N′-diisopropylcarbodiimide, and N,N-dicyclohexylcarbodiimide. In a further embodiment, the coupling agent is N-(3-dimethylaminopropyl)-N′-ethylcarbodiimide hydrochloride. In a further embodiment, ascorbic acid is L-ascorbic acid. In one embodiment of the method to produce Compound (II-A), including any of the foregoing, the amount of hexahydrofarnesoic acid used is about 2 equivalents of the amount of ascorbic acid.

In one embodiment, the method produces Compound (II-B) and comprises contacting hexahydrofarnesoic acid with ascorbic acid, 4-dimethylaminopyridine, N-methyl-2-pyrrolidone, dichloromethane, and a coupling agent. In one embodiment of the method to produce Compound (II-B), the coupling agent is selected from N-(3-dimethylaminopropyl)-N′-ethylcarbodiimide hydrochloride, N,N′-diisopropylcarbodiimide, and N,N′-dicyclohexylcarbodiimide. In a further embodiment, the coupling agent is N-(3-dimethylaminopropyl)-N′-ethylcarbodiimide hydrochloride. In a further embodiment, ascorbic acid is L-ascorbic acid. In one embodiment of the method to produce Compound (II-B), including any of the foregoing, the amount of hexahydrofarnesoic acid used is about 3 equivalents of the amount of ascorbic acid.

In one embodiment, the method produces Compound (II-C) and comprises contacting hexahydrofarnesoic acid with ascorbic acid, 4-dimethylaminopyridine, N-methyl-2-pyrrolidone, ethyl acetate, and a coupling agent. In a further embodiment, the coupling agent is selected from N-(3-dimethylaminopropyl)-N′-ethylcarbodiimide hydrochloride, N,N′-diisopropylcarbodiimide, N,N-dicyclohexylcarbodiimide, and hexafluorophosphate azabenzotriazole tetramethyl uranium. In a further embodiment, the coupling agent is N-(3-dimethylaminopropyl)-N′-ethylcarbodiimide hydrochloride. In a further embodiment, the coupling agent is hexafluorophosphate azabenzotriazole tetramethyl uranium. In a further embodiment, ascorbic acid is L-ascorbic acid. In one embodiment of the method to produce Compound (II-C), including any of the foregoing, the amount of hexahydrofarnesoic acid used is between about 4 and about 6 equivalents of the amount of ascorbic acid. In one embodiment of the method to produce Compound (II-C), including any of the foregoing, the amount of hexahydrofarnesoic acid used is about 5 equivalents of the amount of ascorbic acid.

In another embodiment, the method to produce a compound of Formula (II) comprises:
converting hexahydrofarnesoic acid:

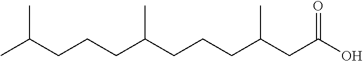 [SMILES: CC(C)CCCC(C)CCCC(C)CC(=O)O]

to hexahydrofarnesyl chloride:

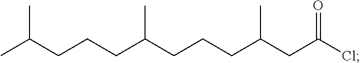 [SMILES: CC(C)CCCC(C)CCCC(C)CC(=O)Cl]

isolating the hexahydrofarnesyl chloride; and
contacting the hexahydrofarnesyl chloride with ascorbic acid and an organic base.

In one embodiment, including any of the foregoing, the ascorbic acid is L-ascorbic acid.

In one embodiment, including any of the foregoing, hexahydrofarnesoic acid is converted to hexahydrofarnesyl chloride in the presence of thionyl chloride (SOCl₂), phosphorus trichloride (PCl₅), phosphorus pentachloride (PCl₃), or oxalyl chloride (ClCOCOCl). In one embodiment, including any of the foregoing, hexahydrofarnesoic acid is converted to hexahydrofarnesyl chloride in the presence of SOCl₂. In one embodiment, including any of the foregoing, the conversion of hexahydrofarnesoic acid to hexahydrofamesyl chloride is conducted in a polar aprotic solvent, for example DCM. In a further embodiment, the conversion is conducted in the presence of a catalytic amount of DMF.

In one embodiment, including any of the foregoing, the organic base is pyridine or a base of the formula NR₃ wherein R is selected independently in each instance from H and alkyl wherein NR₃ typically has at least one, and more often two or three, non-hydrogen R groups. In one embodiment, the organic base is pyridine. In one embodiment, including any of the foregoing, the organic base is DIPEA or NEt₃.

In one embodiment, including any of the foregoing, hexahydrofarnesyl chloride is contacted with ascorbic acid in a polar aprotic solvent, for example DCM or THF. In one embodiment, the polar aprotic solvent is DCM. In a further embodiment, hexahydrofarnesyl chloride is contacted with ascorbic acid in the presence of a catalyst, for example 4-dimethylaminopyridine.

In one embodiment, the method produces Compound (II-A) and comprises:
contacting hexahydrofarnesoic acid with dichloromethane, dimethylformamide, and thionyl chloride to form a reaction mixture that comprises hexahydrofarnesyl chloride;
extracting the hexahydrofarnesyl chloride from the reaction mixture; and
contacting the hexahydrofarnesyl chloride with ascorbic acid, pyridine, DMAP, and dichloromethane.

In one embodiment of the method to produce Compound (II-A), including any of the foregoing, the amount of hexahydrofarnesoic acid used is about 2 equivalents of the amount of ascorbic acid.

In one embodiment, the method produces Compound (II-C) and comprises:
contacting hexahydrofarnesoic acid with dichloromethane, dimethylformamide, and thionyl chloride to form a reaction mixture that comprises hexahydrofarnesyl chloride;
extracting the hexahydrofarnesyl chloride from the reaction mixture; and
contacting the hexahydrofarnesyl chloride with ascorbic acid, pyridine, 4-dimethylaminopyridine, and dichloromethane

In one embodiment of the method to produce Compound (II-C), including any of the foregoing, the amount of hexahydrofarnesoic acid used is between about 4 and about 6 equivalents of the amount of ascorbic acid. In one embodiment of the method to produce Compound (II-C), including any of the foregoing, the amount of hexahydrofarnesoic acid used is about 5 equivalents of the amount of ascorbic acid.

In another aspect, a method is provided to produce Compound (I-C):

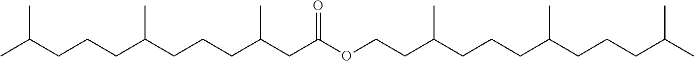 [SMILES: CC(C)CCCC(C)CCCC(C)CCOC(=O)CC(C)CCCC(C)CCCC(C)C]

or a salt and/or stereoisomer thereof.

In one embodiment, the method to produce Compound (I-C) comprises contacting hexahydrofarnesoic acid:

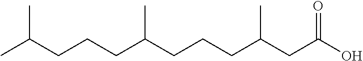 [SMILES: CC(C)CCCC(C)CCCC(C)CC(=O)O]

with hexahydrofarnesol:

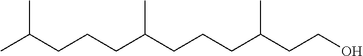 [SMILES: CC(C)CCCC(C)CCCC(C)CCO]

and a coupling agent.

In one embodiment, including any of the foregoing, the coupling agent is selected from N-(3-dimethylaminopropyl)-N′-ethylcarbodiimide hydrochloride (EDC), N,N′-diisopropylcarbodiimide (DIC), and N,N′-dicyclohexylcarbodiimide (DCC). In other embodiments, including any of the foregoing, the coupling agent is selected from hexafluorophosphate azabenzotriazole tetramethyl uranium (HATU), hexafluorophosphate benzotriazole tetramethyl uranium (HBTU), propanephosphonic acid anhydride (T3P), benzotriazole-1-yl-oxy-tris-(dimethylamino)-phosphonium hexafluorophosphate (BOP), benzotriazole-1-yloxytripyrrolidinophosphonium hexafluorophosphate (PyBOP), and 1,1′-carbonyldiimidazole (CDI). In one embodiment, including any of the foregoing, the coupling agent is N-(3-dimethylaminopropyl)-N′-ethylcarbodiimide hydrochloride. In one embodiment, including any of the foregoing, the coupling agent is N,N′-diisopropylcarbodiimide. In one embodiment, including any of the foregoing, the coupling agent is N,N′-dicyclohexylcarbodiimide.

In one embodiment, including any of the foregoing, the method further comprises a catalyst, for example 4-dimethylaminopyridine or 1-hydroxybenzotriazole (HOBT).

In one embodiment, the method to produce Compound (I-C) comprises contacting hexahydrofarnesoic acid with hexahydrofamesol in the presence of N-(3-dimethylaminopropyl)-N′-ethylcarbodiimide hydrochloride and 4-dimethylaminopyridine.

In another embodiment, the method to produce Compound (I-C) comprises:
converting hexahydrofarnesoic acid:

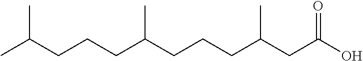 [SMILES: CC(C)CCCC(C)CCCC(C)CC(=O)O]

to hexahydrofarnesyl chloride:

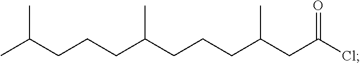 [SMILES: CC(C)CCCC(C)CCCC(C)CC(=O)Cl]

isolating the hexahydrofarnesyl chloride; and
contacting the hexahydrofarnesyl chloride with hexahydrofarnesol:

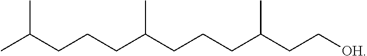 [SMILES: CC(C)CCCC(C)CCCC(C)CCO]

In one embodiment, including any of the foregoing, hexahydrofarnesoic acid is converted to hexahydrofarnesyl chloride in the presence of thionyl chloride (SOCl₂), phosphorus trichloride (PCl₅), phosphorus pentachloride (PCl₃), or oxalyl chloride (ClCOCOCl). In one embodiment, including any of the foregoing, hexahydrofarnesoic acid is converted to hexahydrofarnesyl chloride in the presence of SOCl₂. In one embodiment, including any of the foregoing, the conversion of hexahydrofarnesoic acid to hexahydrofarnesyl chloride is conducted in a polar aprotic solvent, for example DCM. In a further embodiment, the conversion is conducted in the presence of a catalytic amount of DMF.

In one embodiment, including any of the foregoing, hexahydrofarnesyl chloride is contacted with hexahydrofarnesol in a polar aprotic solvent, for example DCM or THF. In one embodiment, the polar aprotic solvent is DCM.

In another aspect, a method is provided to produce a compound of Formula (I):

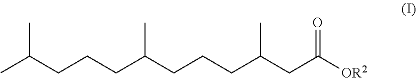

or a salt and/or stereoisomer thereof comprising contacting hexahydrofarnesoic acid:

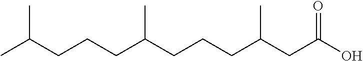 [SMILES: CC(C)CCCC(C)CCCC(C)CC(=O)O]

with R²OH and an acid; wherein R² is selected from a straight or branched C₁₋₆alkyl.

In one embodiment, the acid is selected from phosphoric acid, tosic acid, hydrochloric acid, nitric acid, and sulfuric acid. In one embodiment, the acid is selected from hydrochloric acid, nitric acid, and sulfuric acid. In one embodiment, the acid is sulfuric acid.

In another aspect, a method is provided to produce a compound of Formula (I):

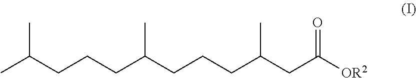

or a salt and/or stereoisomer thereof comprising:
converting hexahydrofarnesoic acid to hexahydrofarnesyl chloride;
isolating the hexahydrofarnesyl chloride; and
contacting the hexahydrofarnesyl chloride with R²OH; wherein R² is selected from a straight or branched C₁₋₆alkyl.

In one embodiment, including any of the foregoing, hexahydrofarnesoic acid is converted to hexahydrofarnesyl chloride in the presence of thionyl chloride (SOCl₂), phosphorus trichloride (PCl₅), phosphorus pentachloride (PCl₃), or oxalyl chloride (ClCOCOCl). In one embodiment, including any of the foregoing, hexahydrofarnesoic acid is converted to hexahydrofarnesyl chloride in the presence of SOCl₂. In one embodiment, including any of the foregoing, the conversion of hexahydrofarnesoic acid to hexahydrofarnesyl chloride is conducted in a polar aprotic solvent, for example DCM. In a further embodiment, the conversion is conducted in the presence of a catalytic amount of DMF.

In one embodiment, including any of the foregoing, hexahydrofarnesyl chloride is contacted with hexahydrofarnesol in a polar aprotic solvent, for example DCM or THF. In one embodiment, the polar aprotic solvent is DCM.

In one embodiment, R² is a straight C₁₋₆alkyl. In one embodiment, R² is a branched C₁₋₆alkyl. In one embodiment, R² is a straight C₁₋₃alkyl. In one embodiment, R² is a straight C₃₋₆alkyl. In one embodiment, R² is a branched C₁₋₃alkyl. In one embodiment, R² is a branched C₃₋₆alkyl. In one embodiment, R² is ethyl. In one embodiment, R² is isopropyl.

In one embodiment, the method produces ethyl-hexahydrofarnesoate:

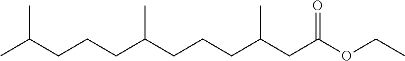 [SMILES: CCOC(=O)CC(C)CCCC(C)CCCC(C)C]

(Compound (I-A)) and comprises contacting hexahydrofarnesoic acid with ethanol and an acid. In a further embodiment, the acid is selected from hydrochloric acid, nitric acid, and sulfuric acid. In a further embodiment, the acid is sulfuric acid.

In one embodiment, the method produces isopropyl-hexahydrofarnesoate:

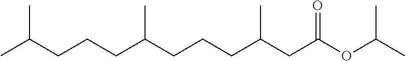 [SMILES: CC(C)CCCC(C)CCCC(C)CC(=O)OC(C)C]

(Compound (I-B)) and comprises contacting hexahydrofarnesoic acid with isopropanol and an acid. In a further embodiment, the acid is selected from hydrochloric acid, nitric acid, and sulfuric acid. In a further embodiment, the acid is sulfuric acid.

In one embodiment, the method produces isopropyl-hexahydrofarnesoate:

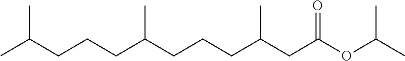 [SMILES: CC(C)CCCC(C)CCCC(C)CC(=O)OC(C)C]

(Compound (I-B)) and comprises:
contacting hexahydrofarnesoic acid with dichloromethane, dimethylformamide, and thionyl chloride to form a reaction mixture;
concentrating the reaction mixture under vacuum; and
adding the concentrated reaction mixture to isopropanol.

In any of the methods described herein for synthesizing a compound of Formula (I) or (II) or Compound (I-A), (I-B), (I-C), (II-A), (II-B), or (II-C), the method further comprises a step of synthesizing hexahydrofarnesoic acid:

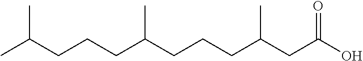 [SMILES: CC(C)CCCC(C)CCCC(C)CC(=O)O]

wherein the synthesis comprises:
contacting hexahydrofarnesol (HHF):

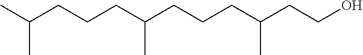 [SMILES: CC(C)CCCC(C)CCCC(C)CCO]

with NaH₂PO₄, TEMPO, and bleach in the absence of solvent; and
adding NaClO₂ over a period between about 4.5 and 6.5 hours.

Also provided herein is process for producing, separating, and purifying hexahydrofarnesoic acid:

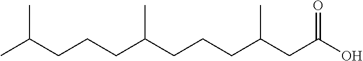 [SMILES: CC(C)CCCC(C)CCCC(C)CC(=O)O]

or a salt and/or stereoisomer thereof comprising:
contacting hexahydrofarnesol (HHF):

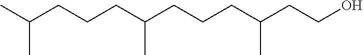 [SMILES: CC(C)CCCC(C)CCCC(C)CCO]

with NaH₂PO₄, TEMPO, and bleach in the absence of solvent; and
adding NaClO₂ over a period between about 4.5 and 6.5 hours.

In one embodiment, including any of the foregoing, the contact of HHF with NaH₂PO₄, TEMPO, and bleach is conducted at a temperature between about −5° C. to about 10° C. In one embodiment, including any of the foregoing, the contact of HHF with NaH₂PO₄, TEMPO, and bleach is conducted at a temperature between about 0° C. to about 5° C.

In one embodiment, including any of the foregoing, the amount of TEMPO is an amount that is between about 0.3 and 0.7 equivalence of the HHF. In one embodiment, including any of the foregoing, the amount of TEMPO is an amount that is about 0.5 equivalence of the HHF.

In one embodiment, including any of the foregoing, the concentration of the NaH₂PO₄ is between about 2.5 M and 3.5 M. In one embodiment, including any of the foregoing, the concentration of the NaH₂PO₄ is about 3.0 M. In one embodiment, including any of the foregoing, the amount of NaH₂PO₄ is an amount that is between about 0.2 and 0.6 equivalence of the HHF. In one embodiment, including any of the foregoing, the amount of NaH₂PO₄ is an amount that is about 0.4 equivalence of the HHF. In one embodiment, including any of the foregoing, the concentration of the NaH₂PO₄ is between about 2.5 M and 3.5 M and the amount is between about 0.2 and 0.6 equivalence of the HHF. In one embodiment, including any of the foregoing, the concentration of the NaH₂PO₄ is about 3.0 M and the amount is about 0.4 equivalence of the HHF.

In one embodiment, including any of the foregoing, the concentration of the bleach is between about 10% v/v and 15% v/v. In one embodiment, including any of the foregoing, the concentration of the bleach is about 12% v/v. In one embodiment, including any of the foregoing, the amount of bleach is an amount that is between about 0.05 and 0.2 equivalence of the HHF. In one embodiment, including any of the foregoing, the amount of bleach is an amount that is about 0.1 equivalence of the HHF. In one embodiment, including any of the foregoing, the concentration of the bleach is between about 10% v/v and 15% v/v and the amount is between about 0.05 and 0.2 equivalence of the HHF. In one embodiment, including any of the foregoing, the concentration of the bleach is about 12% v/v and the amount is about 0.1 equivalence of the HHF.

In one embodiment, including any of the foregoing, the concentration of the NaClO₂ is between about 3 M and about 6 M. In one embodiment, including any of the foregoing, the concentration of the NaClO₂ is between about 4.5 M and about 6 M. In one embodiment, including any of the foregoing, the concentration of the NaClO₂ is about 5 M. In one embodiment, including any of the foregoing, the NaClO₂ is added over a period between about 4.5 and about 5.5 hours. In one embodiment, including any of the foregoing, the NaClO₂ is added over a period of about 5 hours. In one embodiment, including any of the foregoing, the NaClO₂ is added at rates between about 0.5 mL/min and about 1.5 mL/min over the course of the period of between about 4.5 hours and about 6.5 hours. In one embodiment, including any of the foregoing, the NaClO₂ is added over the course of about 4.5 hours and about 6.5 hours at a rate that increases from about 0.5 mL/min and about 1.5 mL/min over the course of addition. In one embodiment, including any of the foregoing, the NaClO₂ is added over the course of about 5 hours at a rate that increases from about 0.7 mL/min and about 1.2 mL/min over the course of the addition. In one embodiment, including any of the foregoing, the concentration of NaClO₂ is about 5 M and the NaClO₂ is added over the course of about 5 hours at a rate that increases from about 0.7 mL/min and about 1.2 mL/min over the course of the addition.

In one embodiment, including any of the foregoing, the method of producing HHFA or a salt and/or stereoisomer thereof comprises:
contacting HHF with a 3.0 M solution of NaH₂PO₄ (0.4 equivalence of the HHF), TEMPO (0.5 equivalence of the II-IF), and a 12% v/v solution of bleach (0.1 equivalence of the HHF) in the absence of solvent at a temperature between about 0° C. to about 5° C.; and
adding NaClO₂ over a period of about 5 hours wherein the NaClO₂ is added at a rate that increases from about 0.7 mL/min and about 1.2 mL/min over the course of the addition.

In a further embodiment, including any of the foregoing, the HHFA is purified using distillation, for example, Kugelrohr distillation.

In a further embodiment, including any of the foregoing, the method affords HHFA and the HHFA is greater than 90% pure. In one embodiment, including any of the foregoing, the HHFA is greater than 92% pure. In one embodiment, including any of the foregoing, the HHFA is greater than 95% pure. In one embodiment, including any of the foregoing, the HHFA is greater than 96%, 97%, 98%, or 99% pure. In one embodiment, the HHFA is 100% pure.

Purification of the compounds of Formula (I) or (II) or compounds (II-A), (II-B), (II-C), (I-A), (I-B), (I-C), or HHFA can be conducted by any method known to a skilled chemist, for example, column chromatography, such as reverse phase column chromatography, normal phase column chromatography, and/or chiral chromatography. Other means of purification include distillation, for example, fractional distillation, vacuum distillation, steam distillation, simple distillation, or azeotropic distillation; crystallization in a solvent or anti-solvent system; or titration.

In any of the methods described herein for synthesizing a compound of Formula (I) or (II) or Compound (I-A), (I-B), (II-A), (II-B), (II-C), or hexahydrofarnesoic acid (HHFA), the method further comprises a step of bio-fermenting beta-farnesene from sugarcane. In certain embodiments, bio-based farnesene is produced from microorganisms, including bioengineered microorganisms, using a renewable carbon source such as sugar. Because bio-based farnesene can be made by fermentation of organic compounds such as sugar by microorganisms, bio-based farnesene is useful in making eco-friendly chemical compounds which can be used for various consumer and industrial product compositions.

In particular embodiments, bio-based farnesene can be produced from fermentation of renewable carbon sources such as sugar using genetically modified microorganisms. In some embodiments, the microorganisms are genetically modified microorganisms in which nucleic acid molecules have been inserted, deleted or modified to produce bio-based farnesene. The methods for producing bio-based farnesene using microorganisms are described in, e.g., U.S. Pat. No. 7,659,097 B2, U.S. Pat. No. 7,399,323 B2, U.S. Pat. No. 7,846,222 B2, U.S. Pat. No. 8,257,957 B2 or International Patent Publication WO2007/139924 A2, each of which is incorporated herein by reference in its entirety.

Bio-based farnesene derived from fermentation of renewable carbons can be used to generate additional hydrocarbon compounds which are suitable in formulations in a variety of consumer or industrial products. For example, farnesol, an alcohol of bio-based farnesene, can be generated via bio-fermentation or generated as previously described (See eg., Chen, W. et al., (2012) European Journal of Medicinal Chemistry, vol. 58, pp. 72-83; and Bennett, C. J. et al., (2004) Bioorganic & Medicinal Chemistry, vol. 12 (9), pp. 2079-2098). Farnesol can be hydrogenated to produce hexahydrofarnesol. The hydroxyl group on the hexahydrofarnesol can then be converted to an acid (hexahydrofarnesoic acid) as described previously (See e.g., Muñoz, L. G., (2012), Synthesis, vol. 44 (06), pp. 862-864, and U.S. Pat. No. 8,603,999 incorporated herein by reference in its entirety). Hexahydrofarnesoic acid can then be used to generate a number of hexahydrofamesene esters which have a number of useful qualities in the formulation of personal care products. Embodiments described herein include the esters dihexahydrofarnesyl ascorbate (Compound (II-A)), trihexahydrofarnesyl ascorbate (Compound (II-B)), tetrahydrofarnesyl ascorbate (Compound (II-C)), hexahydrofarnesyl-hexahydrofarnesoate (Compound (I-AC)), ethyl-hexahydrofarnesoate (Compound (I-A)), and isopropyl-hexahydrofarnesoate (Compound (I-B)). In general, the esters are generated by reacting an alcohol with hexahydrofarnesoic acid. In some embodiments, the reaction is catalyzed by the addition of an additional strong acid. In other embodiments, specific inorganic catalysts such as SO₂Cl₂ and/or dichlorormethane are used.

### Personal Care Products

In certain embodiments, provided herein are compositions for personal care products and other consumer and industrial products. Farnesene derived compounds (e.g., dihexahydrofamesyl ascorbate (Compound (II-A)), trihexahydrofarnesyl ascorbate (Compound (II-B)), tetrahydrofarnesyl ascorbate (Compound (II-C)), hexahydrofarnesyl-hexahydrofarnesoate (Compound (I-AC)), ethyl-hexahydrofarnesoate (Compound (I-A)), and isopropyl-hexahydrofarnesoate (Compound (I-B)) possess many advantageous properties such as solvencies, emollience, spreadability, and/or viscosity, which help to facilitate the application of a personal care product and/or the delivery of an active ingredient. Due to their advantageous properties, the compositions provided herein can be used as a wide range of industrial or consumer products, such as solvents, cleaning products, degreasers, metal cleaners, and other end uses. For example, the compositions are fully compatible to be formulated in hard surface heavy duty cleaners, hand cleaners, graffiti removers, crayon/pen ink removers, bug and tar removers, engine degreasers, laundry pre-spotters, oven cleaners, auto interior cleaners, all-purpose cleaner concentrates and metal cleaning fluids, adhesive removers and paint strippers.

In addition, the compositions are also compatible to be formulated in a wide range of personal care products, such as hair care products (e.g., shampoo, conditioner, anti-frizz treatment, hair repair serum, and the like), lip care products (e.g., lip gloss, lip balm, and the like), skin care products (face serum, face cream, night cream, eye serum, eye cream, moisturizer, and the like), hygiene products (e.g., makeup remover, face cleanser, sanitizing lotion, nail polish remover, and the like), body care products (e.g., body lotion, after shave lotion, and the like), cosmetic makeup products, sun care products (e.g., sun block lotion, sun tan lotion, and the like).

In certain embodiments, the compositions useful as industrial or consumer products (including personal care products) consist essentially of compounds derived from farnesene. In preferred embodiments the personal care products consist essentially of compounds derived sustainably from sugar cane or the waste products of sugar production. In other embodiments, the compositions further comprise one or more additional components to produce end products such as a solvent, a degreaser, a general cleaning product, a metal cleaning product, a personal care product, and the like. In certain embodiments, the compositions further comprise one or more co-solvents or surfactants, or both. In certain embodiments, the compositions further comprise at least one additional component, such as a sensory modifier, a moisturizing agent, an anti-inflammatory agent, a preservative, an anti-aging active, an antioxidant, a co-solvent, surfactant, water, emulsifier, emollient, thickener, or a mixture thereof.

In certain embodiments, the composition can further comprise additives known to the practitioner of skill in the art. Useful additives include, but are not limited to, delaminates, buffering agents, pH control agents, fragrances, perfumes, flavors, essential oils, defoamers, dyes, whiteners, brighteners, solubilizing materials, stabilizers, thickeners, corrosion inhibitors, lotions, mineral oils, enzymes, cloud point modifiers, preservatives, ion exchangers, chelating agents, sudsing control agents, soil removal agents, softening agents, opacifiers, inert diluents, graying inhibitors, stabilizers, polymers, abrasive, exfoliant, and the like, and combinations thereof.

In certain embodiments, one or more additional components/additives incorporated into the present compositions enhance properties or functions of end products. As used herein, the term additional component/additives do not include reactants or reaction products produced by catalytic or hydrogenation reactions of bio-based farnesene. One or more additional components/additives refer to components/additives deliberately added to the compositions for functional purposes. As used herein, the terms “component” and “additive” are used interchangeably, and the same ingredient, e.g., a limonene, may be referred to as a component (e.g., co-solvent) or an additive (e.g., fragrance) depending on its purpose and/amount in the composition.

In certain embodiments, useful sensory modifiers that can be added to the present compositions include, but are not limited to, polyglyceryl-6 octastearate, glyceryl behenate, polyglyceryl-20 octadecabehenate/hydroxystearate, pyrus malus fiber, sunfloweroyl methylglucamide, menthyl nicotinate, dimethicone, dimethicone/vinyl dimethicone crosspolymer, cellulose, glycerin, poly C10-30 alkyl acrylate, and hemisqualane.

In certain embodiments, useful anti-inflammatory agents that can be added to the present compositions include, cannabinoids, Pyrus malus (Apple) Fruit Water (and) Camellia sinensis Leaf (and extract therefrom), Vaccinium macrocarpon (Cranberry) Fruit Extract, Butylene Glycol, Cynanchum atratum Extract, Capryloyl Glycine, Resveratrol, Phragmites communis Extract, Poria cocos Extract, Glycine soja (Soybean) Oil, Arnica montana Flower Extract, Tocopherol, Foeniculum vulgare (Fennel) Fruit Extract, Humulus lupulus (Hops) Extract, Melissa officinalis Leaf Extract, Viscum album (Mistletoe) Leaf Extract, Lactose, and Milk Protein. Useful cannabinoid anti-inflammatory agents include cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT), tetrahydrocannabinol (THC), and tetrahydrocannabinolic acid (THCA). A preferred cannabinoid anti-inflammatory agent is cannabigerol (CBG).

In certain embodiments, useful preservatives that can be added to the present compositions include cannabinoids, ascorbic acid, vitamin E, beeswax, honey, essential oils (e.g., essential oils of lavender, clove, rosemary, tea tree, sage, rosewood, thyme, cinnamon, neem, grape seed, grapefruit seed, and citrus), bentonite clay, copper salts, butylated hydroxyanisole (BHA), butylated hydroxytoluene (BHT), benzoic acid, sodium benzoate, benzyl alcohol, propionic acid, salicylic acid, sorbic acid, calcium sorbate, sodium sorbate, and potassium sorbate. Useful cannabinoid preservative agents include cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT), tetrahydrocannabinol (THC), and tetrahydrocannabinolic acid (THCA). A preferred cannabinoid preservative agent is cannabigerol (CBG).

In certain embodiments, useful antioxidants that can be added to the present compositions include ascorbate esters of hexahydrofarnesoic acid including di-hexahydrofarnesyl ascorbate (Compound (II-A)), tri-hexahydrofarnesyl ascorbate (Compound II-B)), and tetra-hexahydrofarnesyl ascorbate (Compound II-C)), vitamin C, niacinamide, resveratrol, vitamin E, retinol (vitamin A), coenzyme Q10, polyphenols, tocopherol, green tea extracts, curcumin, and flavonoids.

In certain embodiments, useful anti-aging agents that can be added to the present compositions include alpha-lipoic acid, biotin, caffeine, ceramides, coenzyme Q10, glycolic acid, green tea extracts, hyaluronic acid, hydroquinone, jojoba oil, kojic acid, squalane, lactic acid, malic acid, niacinamide, resveratrol, retinol (vitamin A), vitamin C, vitamin E, vitamin K, ascorbate esters of hexahydrofarnesoic acid including di-hexahydrofarnesyl ascorbate, tri-hexahydrofarnesyl ascorbate, and tetra-hexahydrofarnesyl ascorbate.

In certain embodiments, useful co-solvents that can be added to the present compositions include, but are not limited to, saturated hydrocarbon solvents, glycol ethers, fatty acid methyl esters, aliphatic hydrocarbon solvents, acyclic hydrocarbon solvents, halogenated solvents, aromatic hydrocarbon solvents, cyclic terpenes, unsaturated hydrocarbon solvents, halocarbon solvents, polyols, ethers, glycol esters, alcohols, ketones, and any combination thereof. In an embodiment, a composition provided herein further comprises a co-solvent selected from the group consisting of limonene, benzene, toluene, xylene, aromatic high flash aromatic naptha (e.g., aromatic 200), soy methyl ester, ethyl lactate, paraffins (e.g., isopar M), dibasic esters (e.g., DBE-LVP), paraffinic naphthenic solvent, propylene glycol, ethyl alcohol, and mixtures thereof. The addition of such a co-solvent can cause the solvent blend-to-surfactant ratio in the composition to increase.

In certain embodiments, useful surfactants that can be added to the present compositions include, but are not limited to, nonionic surfactants, cationic surfactants, anionic surfactants, amphoteric surfactants, zwitterionic surfactants, or a mixture thereof. Examples of nonionic surfactants include, but are not limited to, one or more of amides such as alkanolamides, ethoxylated alkanolamides, ethylene bisamides; esters such as fatty acid esters, glycerol esters, ethoxylated fatty acid esters, sorbitan esters, ethoxylated sorbitan; ethoxylates such as alkylphenol ethoxylates, alcohol ethoxylates, tristyrylphenol ethoxylates, mercaptan ethoxylates; end-capped and EO/PO block copolymers such as ethylene oxide/propylene oxide block copolymers, chlorine capped ethoxylates, tetra-functional block copolymers; amine oxides such lauramine oxide, cocamine oxide, stearamine oxide, stearamidopropylamine oxide, palmitamidopropylamine oxide, decylamine oxide; fatty alcohols such as decyl alcohol, lauryl alcohol, tridecyl alcohol, myristyl alcohol, cetyl alcohol, ethyl alcohol, stearyl alcohol, oleyl alcohol, linoleyl alcohol and linolenyl alcohol; and alkoxylated alcohols such as ethoxylated lauryl alcohol, trideceth alcohols; and fatty acids such as lauric acid, oleic acid, stearic acid, myristic acid, cetearic acid, isostearic acid, linoleic acid, linolenic acid, ricinoleic acid, elaidic acid, arichidonic acid, myristoleic acid and mixtures thereof. Other examples of non-ionic surfactants include a glycol such as polyethylene glycol (PEG), alkyl PEG esters, polypropylene glycol (PPG) and derivatives thereof. In one embodiment, the surfactant is an alcohol ethoxylate, an alkyl phenol ethoxylate or a terpene alkoxylate.

Examples of cationic surfactants include, but are not limited to, quaternary ammonium compounds, such as cetyl trimethyl ammonium bromide (also known as CETAB or cetrimonium bromide), cetyl trimethyl ammonium chloride (also known as cetrimonium chloride), myristyl trimethyl ammonium bromide (also known as myrtrimonium bromide or Quatemium-13), stearyl dimethyl distearyldimonium chloride, dicetyl dimonium chloride, stearyl octyldimonium methosulfate, dihydrogenated palmoylethyl hydroxyethylmonium methosulfate, isostearyl benzylimidonium chloride, cocoyl benzyl hydroxyethyl imidazolinium chloride, dicetyl dimonium chloride and distearyldimonium chloride; isostearylaminopropalkonium chloride or olealkonium chloride; behentrimonium chloride; as well as mixtures thereof.

Examples of anionic surfactants include, but are not limited to, linear alkylbenzene sulfonates, alpha olefin sulfonates, paraffin sulfonates, alkyl ester sulfonates, alkyl sulfates, alkyl alkoxy sulfates, alkyl sulfonates, alkyl alkoxy carboxylates, alkyl alkoxylated sulfates, monoalkyl phosphates, dialkyl phosphates, sarcosinates, sulfosuccinates, isethionates, and taurates, as well as mixtures thereof. Commonly used anionic surfactants that are suitable as the anionic surfactant component of the composition of the present invention include, for example, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium-monoalkyl phosphates, sodium dialkyl phosphates, sodium lauroyl sarcosinate, lauroyl sarcosine, cocoyl sarcosine, ammonium cocyl sulfate, ammonium lauryl sulfate, sodium cocyl sulfate, sodium trideceth sulfate, sodium tridecyl sulfate, ammonium trideceth sulfate, ammonium tridecyl sulfate, sodium cocoyl isethionate, disodium laureth sulfosuccinate, sodium methyl oleoyl taurate, sodium laureth carboxylate, sodium trideceth carboxylate, sodium lauryl sulfate, potassium cocyl sulfate, potassium lauryl sulfate, monoethanolamine cocyl sulfate, sodium tridecyl benzene sulfonate, and sodium dodecyl benzene sulfonate. Branched anionic surfactants are particularly preferred, such as sodium trideceth sulfate, sodium tridecyl sulfate, ammonium trideceth sulfate, ammonium tridecyl sulfate, and sodium trideceth carboxylate.

Examples of amphoteric surfactants include, but are not limited to, derivatives of aliphatic secondary and tertiary amines in which the aliphatic radical can be straight chain or branched and wherein one of the aliphatic substituents contains from about 8 to about 18 carbon atoms and one contains an anionic water solubilizing group. Specific examples of suitable amphoteric surfactants include the alkali metal, alkaline earth metal, ammonium or substituted ammonium salts of alkyl amphocarboxy glycinates and alkyl amphocarboxypropionates, alkyl amphodipropionates, alkyl amphodiacetates, alkyl arnphoglycinates, and alkyl amphopropionates, as well as alkyl iminopropionates, alkyl iminodipropionates, and alkyl amphopropylsulfonates, such as for example, cocoamphoacetate cocoarnphopropionate, cocoamphodiacetate, lauroamphoacetate, lauroamphodiacetate, lauroamphodipropionate, lauroamphodiacetate, cocoamphopropyl sulfonate caproamphodiacetate, caproamphoacetate, caproamphodipropionate, and stearoamphoacetate.

Examples of zwitterionic surfactants include, but are not limited to, alkyl betaines, such as cocodimethyl carboxymethyl betaine, lauryl dimethyl carboxymethyl betaine, lauryl dimethyl alpha-carboxy-ethyl betaine, cetyl dimethyl carboxymethyl betaine, lauryl bis-(2-hydroxy-ethyl)carboxy methyl betaine, stearyl bis-(2-hydroxy-propyl)carboxymethyl betaine, oleyl dimethyl gamma-carboxypropyl betaine, and lauryl bis-(2-hydroxypropyl)alpha-carboxyethyl betaine, amidopropyl betaines, and alkyl sultaines, such as cocodimethyl sulfopropyl betaine, stearyldimethyl sulfopropyl betaine, lauryl dimethyl sulfoethyl betaine, lauryl bis-(2-hydroxy-ethyl)sulfopropyl betaine, and alkylamidopropylhydroxy sultaines.

In certain embodiments, the compositions provided herein comprise surfactants such as sodium lauryl ether sulfate, ethoxylated alcohol surfactants (e.g., Tomadol 25-3, Tomadol 25-7), fatty acid diethanolamine (e.g., cocamide DEA), orange oil emulsifier (e.g., Videt ME-80), acrylate-based emulsion copolymer (e.g., Alcogum SL-70), polyoxyethers of lauryl alcohol (e.g., Laureth-7), linear isopropylamine dodecylbenzene sulfonate (e.g., Rhodocal IPAM), blended alcohol ethoxylate (e.g., Videt Q3), alkoxylated alcohol (e.g., Tergitol 15-S-7), sodium iminodipropionate (e.g., Amphoteric 400), nonionic alcohol ethoxylates (e.g., Ecosurf EH-6), a palm kernel alcohol ethoxylated and propoxylated surfactant (e.g., Ecosurf SA-7), sodium xylene sulfonate (e.g., Alkatrope SXS-40), or mixtures thereof.

In certain embodiments, useful emulsifiers that can be added to the present compositions include, but are not limited to, polysaccharide ethers, polyglycosides, fatty acids, fatty alcohols, amine oxides, water-soluble cellulose derivatives, alkyl sulfonates, ethoxylated alkyl phenols, alkanaolamides, betaines, zwitterionics, carboxylated alcohols, carboxylic acids, ethoxylated alcohols, and derivatives thereof. In certain embodiments, a composition provided herein further comprises emulsifiers, such as lauryl alcohol (e.g., Laureth-7), fatty acid diethanolamine (e.g., cocamide DEA), ammonium methyl sulfate and fatty alcohol ethoxylate (e.g., Steposol DG), Tomadyne 100 surfactant, linear alcohol (C12-15) ethoxylate, POE-7, POE-3, sodium branched dodecyl benzene sulfonate, or mixtures thereof.

In certain embodiments, useful emollients that can be added to the present compositions include, but are not limited to, conventional lipids materials (e.g., fats, waxes), polar lipids (lipids that have been modified to be more water soluble), silicones, hydrocarbons, and other solvent materials. Emollients can include, for example, petroleum based, fatty acid type, alkyl ethoxylate type, fatty acid ester ethoxylates, fatty alcohol type, polysiloxane type, mucopolysaccharides, or mixtures thereof. Other useful emollients also include polyhydric alcohols, e.g., glycerin and propylene glycol, and the like; polyols such as polyethylene glycols; saccharides and/or polysaccharides, such as sucrose, sorbitol; and urea derivatives such as hydroxyethyl urea and the like. In certain embodiments, the composition provided herein further comprises emollients, such as Crodamol STS (e.g., PPG-3 benzyl ether myristate), Versagel ME-750 (e.g., hydrogenated polyisobutene, butyelen/ethylene/styrene copolymer, ethylene/propylene/styrene copolymer), Softisan 649 (e.g., bis-diglyceryl polyacyladipate-2, Crodamol PTIS (pentaerythrityl tetraisosterate), Super Sterol Ester (e.g., C₁₀₋₃₀ cholesterol/lanosterol esters), or mixtures thereof.

In certain embodiments, useful thickeners that can be added to the present compositions include, but are not limited to, organic thickeners and inorganic thickeners. Organic thickeners may include cellulosic thickeners and their derivatives, natural gums, acrylates, starches, stearates, and fatty acid alcohols. Inorganic thickeners may include clays and salts. Examples of cellulosic thickeners include carboxymethyl hydroxyethylcellulose, cellulose, hydroxybutyl methylcellulose, hydroxyethylcellulose, hydroxypropylcellulose, hydroxypropyl methyl cellulose, methylcellulose, microcrystalline cellulose, sodium cellulose sulfate, and the like. Examples of natural gums include acacia, calcium carrageenan, guar, gelatin, guar gum, hydroxypropyl guar, karaya gum, kelp, locust bean gum, pectin, sodium carrageenan, tragacanth gum, xanthan gum, and the like. Examples of acrylates include potassium aluminum polyacrylate, sodium acrylate/vinyl alcohol copolymer, sodium polymethacrylate, and the like. Examples of starches include oat flour, potato starch, wheat flour, wheat starch, and the like. Examples of stearates include methoxy PEG-22/dodecyl glycol copolymer, PEG-2M, PEG-5M, and the like. Examples of fatty acid alcohols include caprylic alcohol, cetearyl alcohol, lauryl alcohol, oleyl alcohol, palm kernel alcohol, and the like. Some non-limiting examples of clays include bentonite, magnesium aluminum silicate, magnesium trisilicate, stearalkonium bentonite, trimethylamine magnesium aluminum silicate, and the like. Some non-limiting examples of salts include calcium chloride, sodium chloride, sodium sulfate, ammonium chloride, and the like. Some non-limiting examples of thickeners that may be used to thicken the non-aqueous portions of the composition include waxes such as candelilla wax, carnauba wax, beeswax, and the like, oils, vegetable oils and animal oils, and the like. In certain embodiments, the present compositions may further comprise thickeners, such as acrylates C₁₀₋₃₀ cross polymer, Kelzan ASX-T (e.g., xanthan gum), linear alcohol ethoxylate, C₁₂₋₁₄, or mixtures thereof.

In certain embodiments, useful hydrotropes that can be added in the present compositions include, but are not limited to, sodium and ammonium xylene sulfonates, sodium alkyl disulfonates, solvents, particularly alcoholic solvents, such as ethanol, isopropanol, ethoxy diglycol, glycols and polyhydroxy compounds such as propylene glycol, methyl propane, diol, butylene glycol, hexylene glycol, glycerin, dextrose, sorbitol, sucrose, fructose, other sugars, or mixtures thereof.

In certain embodiments, useful pH control agents and/or buffers that can be added to the present compositions include, but are not limited to, sodium hydroxide, potassium hydroxide, tetraethylammonium, sodium citrate, acetic acid, citric acid, hydrochloric acid, and the like. A pH control agent can be added in an amount as needed to keep the composition at a desired pH. Buffers, such as sodium metasilicate, pentahydrate, sodium bicarbonate can also be used to keep the composition at a desired pH. For example, a pH control agent may be added to keep the composition pH selected from about 1 to about 14 depending on the end use of the composition. For example, a composition for heavy duty industrial cleaning application can be formulated to have a pH of about 11 or about 13-14 with a pH control agent and/or buffer. Generally, a pH control agent and/or buffer is added in a small amount in the range of from about 0.1% to about 10%, typically in the range from about 0.5% to about 5%, based on the total weight of the composition.

Depending on end use or application, compounds derived from farnesene can be mixed at any suitable proportions with one or more additional components to produce a composition. In certain embodiments, compounds derived from farnesene may be added as a major weight percent component and one or more additional components may be added to the mixture as a minor weight percent component based on the total weight of the composition. For example, a composition may comprise 69 wt. % of bio-farnesene (or a compound derived from therefrom) and 31 wt. % of at least one additional component (e.g., emulsifier and/or carrier). In certain embodiments, the present composition comprises at least 50, 55, 60, 65, 70, 75, 80, 85, 90, 95, 96, 97, 98, or 99 wt. % of bio-based farnesene or a compound derived therefrom based on the total weight of the composition and at least one additional component. In certain embodiments, the present composition comprises bio-based farnesene or a compound derived therefrom as a minor weight percent component. For example, the present composition comprises less than about 50, 45, 40, 35, 30, 25, 20, 15, 10, 5, 4, 3, 2, or 1 wt. % of bio-based farnesene or a compound derived therefrom and at least one additional component/additive. In certain embodiments, the present composition comprises bio-based farnesene or a compound derived therefrom in any suitable range selected anywhere between about 0.1 wt. % to about 99.9 wt. %, typically between about 0.5 wt. % to about 99.9 wt. %, and at least one additional component, based on the total weight of the composition.

In certain embodiments, the present composition comprises a co-solvent as an additional component in the mixture with bio-based farnesene or a compound derived therefrom. In certain embodiments, a co-solvent can be included in the composition as a major weight percent component of the composition. For example, the composition can comprise at least about 50, 55, 60, 65, 70, 75, 80, 85, 90, 95, 96, 97, 98, or 99 wt. % of co-solvent and bio-based farnesene and/or a compound derived therefrom as a minor component. In other embodiments, the present composition comprises a co-solvent as a minor component. For example, the present composition comprises less than about 50, 45, 40, 35, 30, 25, 20, 15, 10, 5, 4, 3, 2, or 1 wt. % of a co-solvent and any suitable amount of bio-based farnesene and/or a compound derived therefrom. In certain embodiments, the present composition comprises a co-solvent in any suitable range selected anywhere between about 0.1 wt. % to about 99.9 wt. %, typically between about 0.5 wt. % to about 99 wt. %, based on the total weight of the composition. The amount of co-solvent added to the composition depends on end use or application of the composition. For example, in making a solvent blend, bio-based farnesene (or a compound derived therefrom) and co-solvent(s) may be mixed at a ratio of about 70:30, 90:10, 75:25, or any suitable ratios as shown in the Examples section.

In certain embodiments, the present composition comprises compounds derived from farnesene and a surfactant as an additional component. In certain embodiments, one or more surfactants are included in the composition as a major weight percent component. For example, the present composition can comprise one or more surfactants as a major component (e.g., 50 wt. %) of the total weight of the composition. In certain embodiments, the present composition can comprise at least 50, 55, 60, 65, 70, 75, 80, 85, 90, 95, 96, 97, 98, or 99 wt. % of one or more surfactants as a major weight percent component and bio-farnesene (or a compound derived therefrom) as a minor weight percent component based on the total weight of the composition. In other embodiments, one or more surfactants are included in the composition as a minor weight percent component. For example, the present composition can comprise less than about 50, 45, 40, 35, 30, 25, 20, 15, 10, 5, 4, 3, 2, or 1 wt. % of surfactant and any suitable amount of bio-farnesene or a compound derived therefrom. In certain embodiments, the present composition comprises a surfactant in any suitable range selected anywhere between about 0.1 wt. % to about 99.9 wt. %, typically between about 0.5 wt. % to about 99 wt. %, based on the total weight of the composition. The amount of surfactant added to the present composition depends on end use or application of the composition. For example, about 6 wt. % of bio-based farnesene (or a compound derived therefrom) and about 3 wt. % of surfactant can be mixed with other components in making a hand cleaner.

In certain embodiments, the present composition comprises water as at least one additional component in the mixture with one or more compounds derived from farnesene. In certain embodiment, water can be included in the composition as a carrier or diluent. In certain embodiments, water can be included in the composition as a major weight percent component. For example, the present composition can comprise water as a major component (e.g., 83.65 wt. % as a diluent), and bio-based farnesene or a compound derived therefrom as a minor component (e.g., 6.0 wt. %). In certain embodiments, the present composition can comprise at least about 50, 55, 60, 65, 70, 75, 80, 85, 90, 95, 96, 97, 98, or 99 wt. % of water and compounds derived from farnesene as a minor component. In other embodiments, the present composition comprises water as a minor component. For example, the present composition can comprise less than about 50, 45, 40, 35, 30, 25, 20, 15, 10, 5, 4, 3, 2, or 1 wt. % of water and any suitable amount of bio-based farnesene or a compound derived therefrom. In certain embodiments, the present composition comprises water in any suitable range selected anywhere between about 0.1 wt. % to about 99.9 wt. %, typically between about 0.5 wt. % to about 99 wt. %, based on the total weight of the composition. The amount of water added to the composition/product depends on end use or application of the composition. For example, for all purpose cleaner and engine degreaser, water may be added as a major component (e.g., at least 80 wt. % or at least 90 wt. %) and bio-based farnesene or a compound derived therefrom may be added as a minor weight percent component (e.g., less than 10 wt. % or about 1 wt. % or less). In another example, for all-purpose cleaner concentrate, both water and bio-based farnesene (or a compound derived therefrom) may be added as minor weight percent components (e.g., less than 10 wt. % for both).

In certain embodiments, a composition/product comprises a compound derived from farnesene and an emulsifier as an additional component. In certain embodiments, an emulsifier can be included in the composition/product as a major weight percent component of the composition/product. For example, a composition/product can comprise at least about 50, 55, 60, 65, 70, 75, 80, 85, 90, 95, 96, 97, 98, or 99 wt. % of emulsifier and bio-based farnesene (or a compound derived therefrom) as a minor component. In certain embodiments, one or more emulsifiers are included in the composition/product as a minor component. For example, a composition/product can comprise less than about 50, 45, 40, 35, 30, 25, 20, 15, 10, 5, 4, 3, 2, or 1 wt. % of emulsifier and any suitable amount of the compound derived from farnesene. In certain embodiments, a composition/product comprises one or more emulsifiers in any suitable range selected anywhere between about 0.1 wt. % to less than about 50 wt. %, typically between about 1 wt. % to less than about 50 wt. %, based on the total weight of the composition. The amount of emulsifier(s) added to the composition/product depends on end use or application of the composition/product. For example, about 69 wt. % of bio-based farnesene (or a compound derived therefrom) and about 11 wt. % of emulsifier can be mixed with other components in making a solvent degreaser and ink remover as shown in the examples section.

In certain embodiments, a composition/product comprises a compound derived from farnesene and an emollient as an additional component. In certain embodiments, an emollient can be included in the product as a major weight percent component of the composition/product. For example, a composition/product can comprise at least about 50, 55, 60, 65, 70, 75, 80, 85, 90, 95, 96, 97, 98, or 99 wt. % of emollient and a compound derived from farnesene as a minor component. In certain embodiments, one or more emollients are included in the composition/product as a minor component. For example, a composition/product can comprise less than about 50, 45, 40, 35, 30, 25, 20, 15, 10, 5, 4, 3, 2, or 1 wt. % of emollient and any suitable amount of bio-based farnesene (or a compound derived therefrom). In certain embodiments, a composition/product comprises one or more emollients in any suitable range selected anywhere between about 0.1 wt. % to less than about 50 wt. %, typically between about 1 wt. % to less than about 50 wt. %, based on the total weight of the composition. The amount of emollient(s) added to the composition/product depends on end use or application of the composition/product.

In certain embodiments, a composition/product comprises a compound derived from farnesene and a thickener as an additional component. In certain embodiments, a thickener can be included in the product as a major weight percent component of the composition/product. For example, a composition/product can comprise at least about 50, 55, 60, 65, 70, 75, 80, 85, 90, 95, 96, 97, 98, or 99 wt. % of thickener and bio-based farnesene (or a compound derived therefrom) as a minor component. In certain embodiments, one or more thickeners are included in the composition/product as a minor component. For example, a composition/product can comprise less than about 50, 45, 40, 35, 30, 25, 20, 15, 10, 5, 4, 3, 2, or 1 wt. % of thickener and any suitable amount of bio-based farnesene (or a compound derived therefrom). In certain embodiments, a composition/product comprises one or more thickeners in any suitable range selected anywhere between about 0.1 wt. % to less than about 50 wt. %, typically between about 1 wt. % to less than about 50 wt. %, based on the total weight of the composition. The amount of thickener(s) added to the composition/product depends on end use or application of the composition/product. For example, about 90 wt. % of the compound derived from farnesene and about 10 wt. % thickener can be mixed together to make a metal cleaner.

The proportions of components described above are exemplary, and one or more components and/or additives described herein can be mixed together at suitable proportions to provide desired properties to the composition/product.

The compositions/products of the present invention can be manufactured through typical processes such as mixing or blending the composition. Some or all of the ingredients can be mixed together at once, or in some embodiments, the compositions can be prepared through the sequential addition of ingredients to the mixing vessel with low or moderate shearing mixing with order of addition and temperature suitable for the selected ingredients.

In certain embodiments, the end product is in the form of a wipe which is impregnated with the present compositions. The wipe may be in any suitable form, such as nonwoven material, cloth, sponge, or any absorbent material which can be pre-moistened with the present compositions. In some embodiments, the end product may be stored in a container with an applicator, such as a spray nozzle.

The compositions can be used in any method deemed suitable by the practitioner of skill. In certain embodiments, provided herein are methods of cleaning. The methods comprise the step of contacting a substrate with a sufficient amount of a composition and/or a product provided herein to clean the substrate. In certain embodiments, provided herein are methods of degreasing. The methods comprise the step of contacting a substrate with a sufficient amount of a composition and/or a product provided herein to degrease the substrate. Useful substrates include, but are not limited to, domestic and commercial surfaces, skin, hands, floors, walls, engines, clothing, ovens, automobiles, automobile interiors, metals, metal parts, and any other substrate deemed suitable by the practitioner of skill.

In certain embodiments, provided are methods of treating or conditioning skin, hair, or nails. The methods comprise the step of applying to the skin, hair, nails, or any other suitable substrates with a sufficient amount of a composition and/or a product provided herein to enhance appearance and/or other properties of the skin, hair, or nails.

In certain embodiments, a kit is provided herein with the present compositions. The kit may comprise the present compositions described herein and instructions for using the composition and/or product. For example, the kit embodiment may include instructions for using the composition neat or instructions for diluting the composition with water (or other suitable diluent) and the appropriate dilution ratio. The kit may further comprise a wipe that is dry or pre-moistened with the present compositions, gloves, or other accessory items.

### EXAMPLES

Beta-farnesene was prepared from sugarcane by fermentation of recombinant yeast strains in a culture medium containing sugarcane syrup. As described in the examples below, beta-farnesene served as the source to make a number of novel compounds that have value as ingredients in cosmetics, including the compounds described herein.

### Example 1: Synthesis of Hexahydrofarnesol and Hexahydrofarnesoic Acid

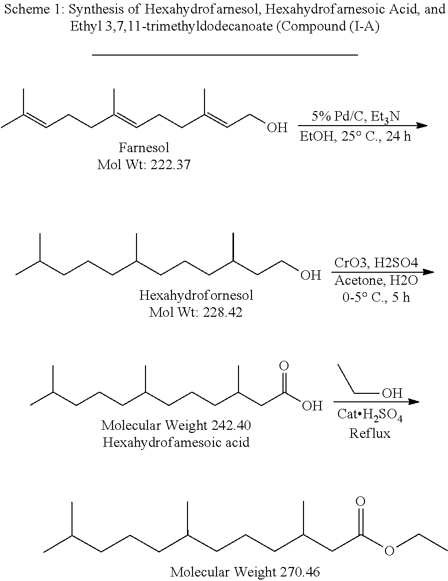

Hexahydrofarnesol was prepared as previously described. (See eg., Chen, W. et al., (2012) European Journal of Medicinal Chemistry, vol. 58, pp. 72-83; and Bennett, C. J. et al., (2004) Bioorganic & Medicinal Chemistry, vol. 12 (9), pp. 2079-2098. In brief, farnesol (106.68 g, 0.48 mole), triethylamine (TEA, 4.94 g, 0.049 mole), ethanol (300 mL) and 5% Pd-C (21.63 g, 0.2 wt %) were charged into a 1 L pressure reactor. The reagents and solvent were mixed under hydrogen pressure at 365 psi stirring at 200 RPM at room temperature (RT). The temperature was gradually increased to 50° C. and the slurry was stirred for 16 h. The reaction mixture was filtered through a bed of diatomaceous earth (CELITE) under a nitrogen blanket. The CELITE bed was washed with 600 mL isopropanol and the mother liquor was concentrated by vacuum rotoevaporation of solvent. The concentrated mother liquor was then dissolved in 350 mL of ethyl acetate (EtOAc) and washed twice with 200 mL of brine. The organic phase was then dried with magnesium sulfate and filtered. The filtrate was washed with 150 mL of EtOAc. The filtered solution was concentrated by vacuum rotoevaporation to afford a clear, colorless oil of hexahydrofamesol (HHF). Yield was 98.6 g (90%).

¹HNMR Spectrum (400 MHz, CDCl₃): 3.75-3.63 (m, 2H), 1.65-1.55 (m, 4H), 1.50-1.24 (m, 10H), 1.15-1.02 (m, 4H), 0.92-0.83 (m, 12H)

Hexahydrofarnesoic acid was prepared by oxidizing hexahydrofarnesol using Jones reagent. (See e.g., Munoz, L. G., (2012), Synthesis, vol. 44 (06), pp. 862-864, and U.S. Pat. No. 8,603,999 incorporated herein by reference in its entirety).

In an Erlenmeyer flask, chromium (VI) oxide (36 g, 2 eq) was dissolved in water (130 mL) and was chilled in an ice bath. Sulfuric acid (40 mL) was added slowly to chromium solution to a dark reddish-orange solution. HHF (40.8 g) and acetone (600 mL) were charged into a 2 L 3-neck glass round bottom flask. The reaction mixture was stirred at 214 RPM with an overhead stirrer while the round bottom flask was immersed in an ice chill bath to maintain 0-5° C. Jones reagent was added to the reaction mixture dropwise to avoid exotherm above 8° C. over 2.5 hours. After completion of the addition, the reaction mixture was allowed to stir an additional 6 hours at RT. The reaction mixture was then cooled down to below 10° C. 200 mL of IPA was added slowly to quench the excess Jones reagent. After completion of quenching, the green reaction was filtered, and the mother liquor was concentrated under vacuum. Hexahydrofarnesoic acid was extracted from the mother liquor using 200 mL brine and 200 mL EtOAc. The aqueous layer was washed with 200 mL EtOAc twice. All organic layers were combined and dried using magnesium sulfate and concentrated. Hexahydrofarnesoic acid was recovered through distillation as described below.

Lab scale Distillation Procedure

Crude Hexahydrofarnesoic acid (HHFA) mixture (80-86% area) was heated from 130° C. to 155° C. under vacuum at 0.45-1 Torr to remove light boiling impurities. The distillation temperature was gradually increased from 156° C. to 165° C. at a vacuum at 0.2-0.4 Torr and the distillate was collected to afford HHFA (>92% area purity). The distillation product yield was greater than 85% depending on the distillation condition used.

TABLE 1
Results of the distillation of crude HHFA reaction mixture using lab scale distillation unit.

| | HHFA | | | | | HHFA | | HHFA | HHFA |
|---|---|---|---|---|---|---|---|---|---|
| HHFA | Crude | Mass of | | Lights Cut | HHFA | Distillate | HHFA | Distillation | Distillation |
| crude | Purity | Light | Pressure | Temperature | Distillate | Purity | Distillate | Temperature | Pressure |
| (g) | (% area) | cuts (g) | (Torr) | (C.) | (g) | (% area) | Yield (%) | (C.) | (Torr) |
| 22.8 | 86.7 | 0.3 | 0.5 | 145 | 19.2 | 92 | 92 | 147-160 | 0.2 |
| 28.8 | 87.5 | 0.8 | 0.5 | 147 | 23.2 | 93 | 92 | 147-157 | 0.2 |
| 24.9 | 81.4 | — | 0.5 | 147 | 19.9 | 95 | 98 | 155-158 | 0.2 |
| 20.6 | 80.2 | 0.5 | 0.3 | 147 | 18.2 | 91 | 96 | 147-157 | 0.3 |
| 22.8 | 86.7 | 0.3 | 0.4 | 145 | 19.7 | 92 | 92 | 153-158 | 0.3 |
| 22.6 | 89.8 | — | 0.5 | 148 | 20.8 | 97 | 99 | 150-158 | 0.2 |
| 112.1 | 87 | 4.5 | 1 | 155 | 95.3 | 96.8 | 98 | 165 | 0.4 |

¹HNMR Spectrum (400 MHz, DMSO-d6). 11.95 (s, 1H), 2.21-2.15 (m, 1H), 2.02-1.96 (m, 1H), 1.85-1.77 (m, 1H), 1.56-1.46 (m, 1H), 1.33-1.01 (m, 15H), 0.88-0.82 (m, 13H)

### Example 2: Synthesis of Ethyl 3,7,11-trimethyldodecanoate (Ethyl hexahydrofarnesoate, Compound (I-A))

25 g of hexahydrofarnesoic acid and 75 g of ethanol were transferred into a flask and 0.5 mL of concentrated sulfuric acid was added. The solution was refluxed for 8 h and the reaction was followed by GCMS. Once all HHFA was consumed the mixture was concentrated and extracted using 200 mL of MTBE and brine (200 mL). The aqueous layer was washed one more time with 200 mL of MTBE. All the organic layers were combined and dried under anhydrous sodium sulfate. The slurry was filtered and concentrated to obtain 25.5 g (91% yield) of ethyl hexahydrofarnesoate.

### Example 3: Synthesis of Isopropyl 3,7,11-trimethyldodecanoate (Isopropyl hexahydrofarnesoate, Compound (I-B))

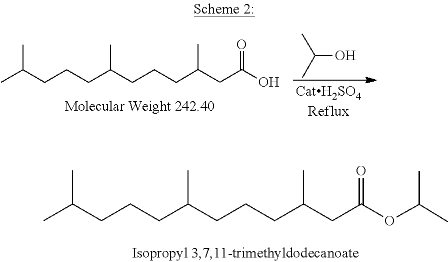

10 g of hexahydrofarnesoic acid and 65 g of isopropanol were transferred into a flask and 0.5 mL of concentrated sulfuric acid was added. The solution was refluxed for 48 h and the reaction was followed by GCMS. Once all HHFA was consumed the mixture was concentrated and extracted using 200 mL of MTBE and brine (100 mL). The aqueous layer was washed one more time with 50 mL of MTBE. All the organic layers were combined and dried under anhydrous sodium sulfate. The slurry was filtered and concentrated to obtain 10.5 g (90% yield) of isopropyl hexahydrofarnesoate.

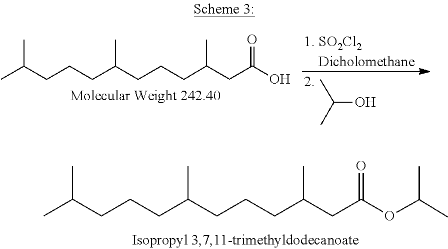

Hexahydrofarnesoic acid (2.06 g, 0.0085 mol), dichloromethane (7.50 ml) and catalytic amount of dimethylformamide (DMF, 0.2 mL) were added into a 50 mL round bottom flask under nitrogen atmosphere at 20-25° C. Thionyl chloride (1.35 g) was added at 20-25° C. The reaction mixture was stirred for 2 h and progress of the reaction was monitored by GCMS. After completion, the reaction mixture was concentrated under vacuum and dissolved in dichloromethane (2.5 mL). Isopropanol (0.75 g, 3 eq) was added in a round bottom flask under nitrogen atmosphere and was cooled to 0° C. Prepared DCM solution of hexahydrofarnesyl chloride was added to cold isopropanol dropwise. The resulting mixture was stirred at 0-5° C. for 1 h then allowed to 20-25° C. for 8 h. Progress of the reaction was monitored by GCMS. Upon completion of the reaction, water (25 mL) was added to the reaction mixture, extracted using ethyl acetate (2×50 mL). The combined organic layers were washed with 0.5 N HCl (20 mL), water (20 mL) then brine (10 mL). The organic layer dried over Na₂SO₄, filtered, concentrated under vacuum at 40-45° C. to obtain a crude. The crude was purified by column chromatography using heptane and ethylacetate to obtain 1.9 g of isopropyl hexahydrofarnesoate (yield 80%).

### Example 4: Synthesis of 3,7,11-trimethyldodecyl 3,7,11-trimethyldodecanoate (Hexahydrofarnesyl-hexahydrofarnesoate, Compound (I-C))

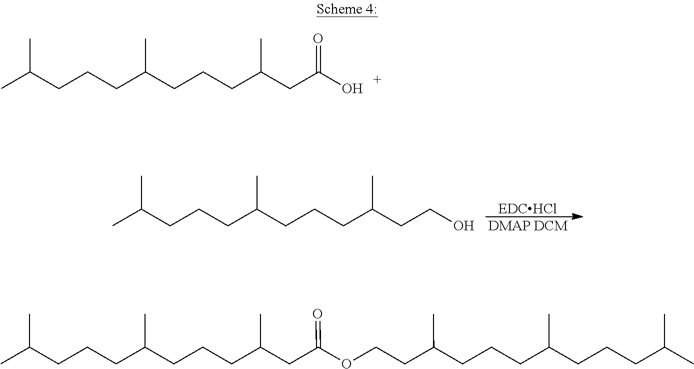 [SMILES: CC(C)CCCC(C)CCCC(C)CC(=O)O ; CC(C)CCCC(C)CCCC(C)CCO ; CC(C)CCCC(C)CCCC(C)CCOC(=O)CC(C)CCCC(C)CCCC(C)C]

Hexahydrofarnesoic acid (10 g, 0.041 mol), hexahydrofarnesol (9.43 g, 1 eq), N-(3-dimethylaminopropyl)-N′-ethylcarbodiimide hydrochloride (EDC.HCl, 7.9 g, 1 eq), and 4-dimethylaminopyridine (DMAP, 503 mg, 0.1 eq) were dissolved in 20 mL of dichloromethane (DCM) under nitrogen. The resulting mixture was stirred for 10 h and the reaction was followed by GCMS. After completion, the mixture was washed with water (50 mL) and extracted using 50 mL of DCM. The organic layer was further washed with 100 mL of brine solution and dried under anhydrous sodium sulfate. The slurry was filtered, and the mother liquor was concentrated to obtain a crude oil which was purified by column chromatography using heptane to recover 14.7 g of hexahydrofarnesyl-hexahydrofarnesoate (yield 76%).

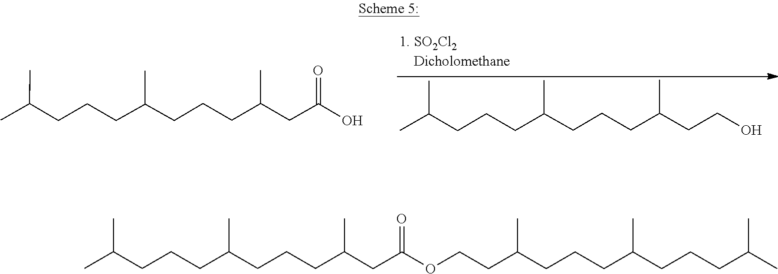 [SMILES: CC(C)CCCC(C)CCCC(C)CC(=O)O ; CC(C)CCCC(C)CCCC(C)CCO ; CC(C)CCCC(C)CCCC(C)CCOC(=O)CC(C)CCCC(C)CCCC(C)C]

Hexahydrofamesoic acid (2.06 g, 0.0085 mol), dichloromethane (7.50 ml) and a catalytic amount of dimethylformamide (0.2 mL) were added into a 50 mL round bottom flask under nitrogen atmosphere at 20-25° C. Thionyl chloride (1.35 g) was added at 20-25° C. The reaction mixture was stirred for 2 h and progress of the reaction was monitored by GCMS. After completion residual thionyl chloride was evaporated under vacuum and the reaction mixture was dissolved in dichloromethane (2.5 mL). Hexahydrofarnesol (2.13 g, 1.1 eq) was added in a round bottom flask under nitrogen atmosphere and was cooled to 0° C. Prepared hexahydrofarnesyl chloride, dissolved in dichloromethane (2.50 mL) was added to cold hexahydrofarnesol dropwise. The resulting reaction mixture was stirred at 0-5° C. for 1 h then allowed warm to room temperature for 8 h. Progress of the reaction was monitored by GCMS. Upon completion, water (25 mL) was added to the reaction mixture and the product was extracted using ethyl acetate (2×50 mL). The combined organic layers were washed with 2N HCl (20 mL), water (20 mL) then brine (10 mL). The organic layer dried over Na₂SO₄, filtered, concentrated to obtain a crude which was purified by column chromatography with a gradient of heptane and ethylacetate and recovered 2.7 g of Hexahydrofarnesyl-hexahydrofarnesoate (yield ~70%).

¹H-NMR (CDCl₃, 400 MHz): 4.07 (m, 2H), 2.26 (m, 1H), 2.06 (m, 1H), 1.90 (m, 1H), 1.60 (m, 2H), 1.48 (m, 2H), 1.21 (m, 22H), 1.03 (m, 5H), 0.83 (m, 24H).

### Example 5: (Vitamin C Conjugates) Synthesis of Dihexahydrofarnesyl ascorbate (1S)-1-(3,4-dihydroxy-5-oxo-2,5-dihydrofuran-2-yl)ethane-1,2-diyl bis(3,7,11-trimethyldodecanoate) (Compound (II-A))

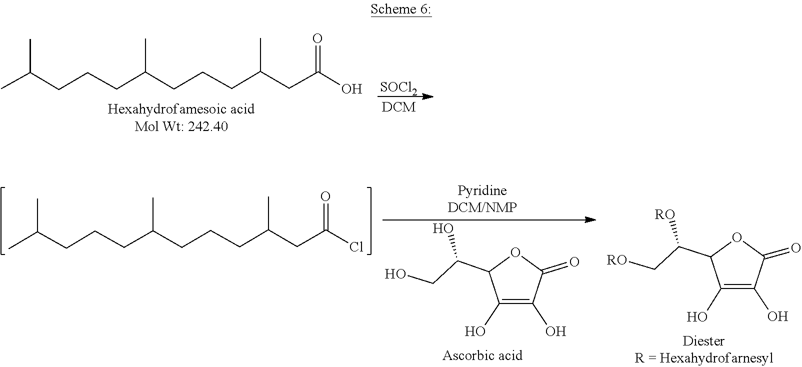

Hexahydrofarnesoic acid (6.88 g, 0.028 moles, 2 eq), Dichloromethane (20 mL) and a catalytic amount of DMF (0.1 mL) were added into a 50 mL round bottom flask under nitrogen atmosphere and cooled to 4° C. Thionyl chloride (1M in DCM, 56 mL, 4 eq) was added slowly over 80 minutes. The reaction mixture was stirred at 0-5° C. for 1 h and at room temperature for 3 h. Progress of the reaction was monitored by GCMS. After completion, the solvent and residual thionyl chloride were evaporated and the crude hexahydrofarnesyl chloride oil was dissolved in dichloromethane (15 mL). Ascorbic acid (2.5 g, 1 eq), Pyridine (11.22 g, 10.0 eq), DMAP (0.173 g, 0.10 eq) and Dichloromethane (37.5 mL, 15 vol) were added in 100 mL round bottom flask under nitrogen atmosphere at 20-25° C. The suspension was cooled to 0° C. and hexahydrofarnesyl chloride was added over 60 mins. The resulting reaction mixture was stirred at 0-5° C. for 4 h and at room temperature for 16 h. Progress of the reaction was monitored by LCMS. Upon completion, water (25 mL) was added to the reaction mixture, extracted using Ethyl acetate (2×50 mL). The combined organic layers was washed with 2N HCl (20 mL), water (20 mL) then brine (30 mL). Organic layer was dried over Na₂SO₄, filtered, evaporated under vacuum to obtain a crude as light brown oil. The crude was purified by C18 column chromatography by using acetonitrile and water to get dihexahydrofarnesyl ascorbate (yield 20%). LCMS: ESI, m z 624.46

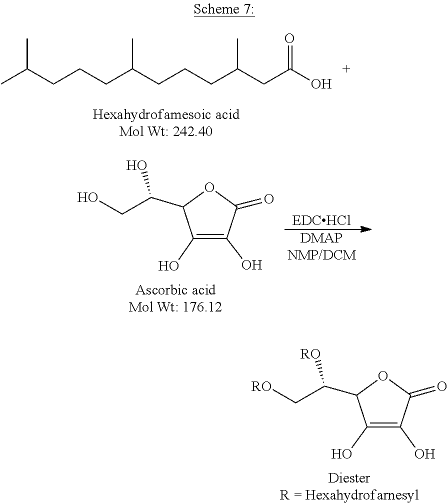

Ascorbic acid (3.6 g, 1 eq), hexahydrofarnesoic acid (15 g, 3 eq), N-(3-dimethylaminopropyl)-N′-ethylcarbodiimide hydrochloride (EDC.HCl, 9.1 g, 3.5 eq), and 4-dimethylaminopyridine (DMAP, 1.26 g, 0.5 eq) were dissolved in 100 mL of N-methyl-2-pyrrolidone (NMP) under nitrogen. The resulting mixture was stirred for 24 h, and the reaction was followed by LCMS. After completion, the mixture was dissolved in 100 mL EtOAc and washed with water (150 mL). The organic layer was further washed with 100 mL of brine solution and dried under anhydrous sodium sulfate. The slurry was filtered, and the mother liquor was concentrated to obtain a crude oil which was purified by C18 column chromatography by using acetonitrile and water to get dihexahydrofarnesyl ascorbate.

### Example 6: (Vitamin C Conjugates) Synthesis of Trihexahydrofarnesyl ascorbate (1S)-1-(4-hydroxy-5-oxo-3-((3,7,11-trimethyldodecanoyl)oxy)-2,5-dihydrofuran-2-yl)ethane-1,2-diyl bis(3,7,11-trimethyldodecanoate) (Compound II-B))

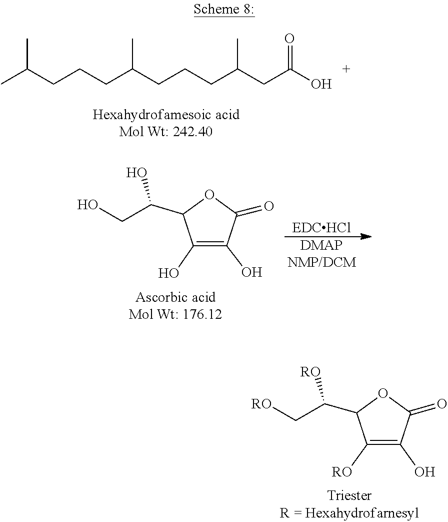

Ascorbic acid (0.727 g, 0.004 mol, 1 eq), hexahydrofarnesoic acid (3 g, 0.012 mol, 3 eq), N-(3-dimethylaminopropyl)-N′-ethylcarbodiimide hydrochloride (EDC.HCl, 2.77 g, 3.5 eq), and 4-dimethylaminopyridine (DMAP, 0.25 g, 0.5 eq) were dissolved in 20 mL of N-Methyl-2-pyrrolidone (NMP) and 10 mL of dichloromethane (DCM) under nitrogen. The resulting mixture was stirred for 24 h and the reaction was followed by LCMS. After completion, the mixture was washed with 0.5 M hydrochloric acid (50 mL) and extracted using 50 mL of DCM. The organic layer was further washed with 100 mL of brine solution and dried under anhydrous sodium sulfate. The slurry was filtered, and the mother liquor was concentrated to obtain a crude oil which was purified by column chromatography using heptane and ethylacetate to recover 1.04 g of trihexahydrofarnesyl ascorbate (yield 30%).

¹H-NMR (CDCl₃, 400 MHz): 5.51-5.47 (m, 1H); 4.97-4.96 (d, 1H, J=2.0 Hz); 4.41-4.37 (dd, 1H, J=11.6, 5.6 Hz); 4.31-4.26 (ddd, 1H, J=11.2, 6.8, 2.4 Hz), 2.64-2.53 (m, 1H); 2.35-2.29 (m, 3H); 2.16-2.00 (m, 3H); 1.98-1.80 (m, 2H); 1.57-1.47 (septet, 3H, J=6.4 Hz); 1.41-0.83 (m, 77H).

LCMS: ESI, m/z 848.67

### Example 7: (Vitamin C Conjugates) Synthesis of Tetrahexahydrofarnesyl ascorbate 2-((1S)-1,2-bis((3,7,11-trimethyldodecanoyl)oxy)ethyl)-5-oxo-2,5-dihydrofuran-3,4-diyl bis(3,7,11-trimethyldodecanoate) (Compound II-C))

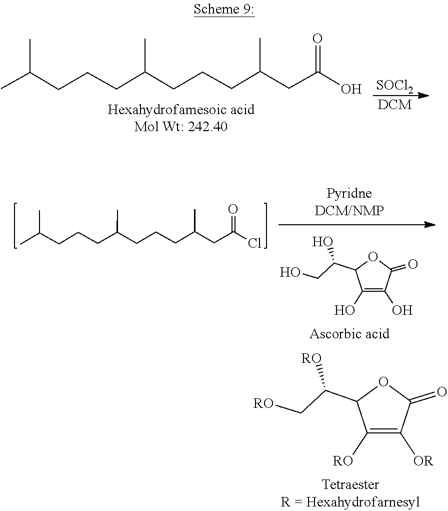

Hexahydrofarnesoic acid (1.72 g, 5.00 eq), dichloromethane (7.50 ml, 30.0 volumes) and catalytic amount of DMF were added into a 50 mL round bottom flask under nitrogen atmosphere at 20-25° C. Thionyl chloride (1.35 g, 0.0112 mol, 8.00 eq) was added at 20-25° C. The reaction mixture was stirred at 20-25° C. for 2 h and progress of the reaction was monitored by GCMS. After completion, the reaction was concentrated under vacuum and dissolved in dichloromethane (1.25 mL, 5 vol). Ascorbic acid (0.25 g, 0.0014 mol, 1.00 eq), pyridine (1.12 g, 0.014 mol, 10.0 eq), DMAP (0.016 g, 0.14 mmol, 0.10 eq) and dichloromethane (3.75 mL, 15 vol) were added in 100 mL round bottom flask under nitrogen atmosphere. The reaction was cooled to 0° C. and hexahydrofarnesyl chloride was slowly added to it. The resulting reaction mixture was stirred at 0-5° C. for 4 h then allowed to room temperature for 4 h. Progress of the reaction was monitored by LCMS. Upon completion, water (25 mL) was added to the reaction mixture and the product was extracted using ethyl acetate (2×50 mL). The combined organic layers were washed with 2N HCl (20 mL), water (20 mL) then brine (10 mL). Organic layer dried over Na₂SO₄, filtered, evaporated the solvent under vacuum at 40-45° C. to obtain crude oil. The crude was purified by silica gel using heptane and dichloromethane to get Tetrahexahydrofarnesyl ascorbate (yield 20%). ¹H-NMR (CDCl₃, 400 MHz): 5.50-5.48 (dd, 1H, J=7.2, 6.0 Hz); 5.41-5.40 (d, 1H, J=5.6 Hz); 4.42-4.38 (dd, 1H, J=11.6, 5.6 Hz); 4.30-4.25 (dd, 1H, J=11.6, 8.0 Hz); 2.51-2.47 (m, 2H); 2.34-2.26 (m, 4H); 2.13-2.07 (m, 2H); 1.99-1.86 (m, 4H); 1.55-1.47 (septet, 4H, J=6.4 Hz); 1.35-1.24 (m, 37H); 1.15-1.07 (m, 19H); 1.00-0.98 (m, 6H); 0.93-0.91 (d, 3H, J=6.4 Hz); 0.87-0.83 (m, 39H). LCMS: ESI, m z 1090.17 [M+NH4]+

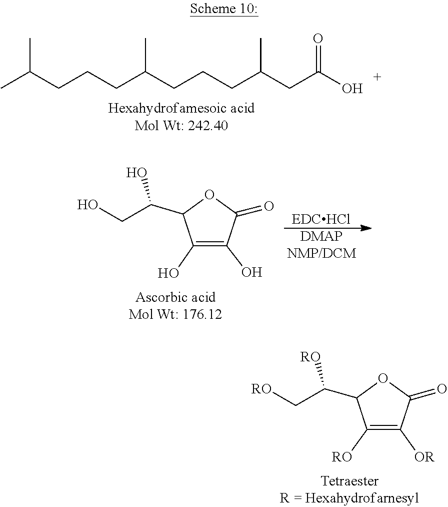

Ascorbic acid (0.727 g, 0.004 mol, 1 eq), hexahydrofarnesoic acid (5 g, 0.012 mol, 5 eq), N-(3-dimethylaminopropyl)-N′-ethylcarbodiimide hydrochloride (EDC.HCl, 4.35 g, 5.5 eq), and 4-dimethylaminopyridine (DMAP, 0.25 g, 0.5 eq) were dissolved in 20 mL of N-methyl-2-pyrrolidone (NMP) under nitrogen. The resulting mixture was stirred for 24 h and the reaction was followed by LCMS. After completion, 50 mL ethyl acetate was added, and the reaction was stirred for 15 min. The mixture was extracted from water (50 mL). The organic layer was further washed with 100 mL of brine solution and dried under anhydrous sodium sulfate. The slurry was filtered, and the mother liquor was concentrated to obtain a crude oil which was purified by column chromatography using heptane and dichloromethane to recover tetrahexahydrofarnesyl ascorbate.

### Example 8: HDD Physico-Chemical Evaluations Vs. Benchmarks: PROTOL-Mineral Oil, and Squalane

Various physical properties of HDD were compared to two cosmetic ingredient benchmarks: PROTOL mineral oil (hereinafter “PROTOL”) and squalane. PROTOL mineral oil was chosen as a benchmark because it has a close dynamic viscosity value to HDD and squalane was chosen because it has the same carbon number (C30) as HDD and a somewhat similar chemical structure.

### Viscosity Values

| Material | Viscosity @20 C., cP |
|---|---|
| HDD | 61.5 |
| PROTOL | 64.6 |
| Squalane | 23.3 |

Unexpectedly, despite having the same number of carbons, HDD has almost double the viscosity value of squalane.

### Surface Tension Values

The surface tension of a material in beauty and personal care (B&PC) ingredients accounts for many of the sensory attributes. The lower the surface tension of a material the better is the material's spreadability, slip, glide, and lubricity.

| Material | Surface tension, mN/m |
|---|---|
| HDD | 24.5 |
| PROTOL | 34.2 |
| Squalane | 26.1 |

Both HDD and squalane have lower surface tension values than PROTOL which is attributed to their more pleasant sensory performances.

Static water contact angle is another indication of material spreading and wetting.

| Material | Water contact angle |
|---|---|
| HDD | 19.8 |
| PROTOL | 43.9 |
| Squalane | 30.9 |

The highest value of contact angle on glass was observed for PROTOL while HDD had the lowest contact angle value.

Gloss is a very desirable property in B&PC products. Gloss was measured at 20 deg.

| Material | Gloss |
|---|---|
| HDD | 70.6 |
| PROTOL | 70.7 |
| Squalane | 65.7 |

Both HDD and PROTOL have close values for gloss which are somewhat higher than squalane.

Dynamic viscosities by Capillary viscometer and by a rheometer vs benchmarks: the closest benchmark is ERVOL white mineral oil. HDD-69.5, Squalane-28.3, ERVOL-47.1 cP @25C. The higher viscosity of HDD may help to better position and differentiate it from Squalane.

### Example 9: Wetting on Hydrophobic-PTFE and Hydrophilic-Glass Slide Substrates by Static Contact Angle and Surface Tension by Pendant Drop Shape Analysis

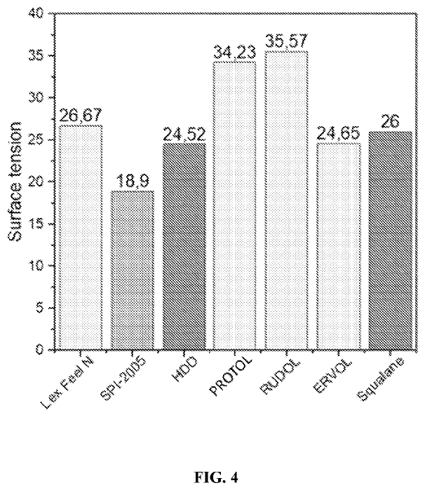

The wetting values (on hydrophobic-PTFE and on hydrophilic-glass slides) were measured and compared for amodimethicone, LEX FEEL (diheptyl succinate), non-redistilled HDD, distilled HDD, PROTOL (mineral oil), RUDOL (mineral oil), ERVOL (mineral oil), and squalane. As expected, amodimethicone had the best wetting on both substates and had the lowest surface tension. Non-redistilled HDD together with RUDOL and PROTOL mineral oils show the highest values of surface tension. Distilled HDD has lower surface tension value, comparable to squalane, ERVOL (mineral oil), and LEX FEEL, see FIG. 4. The lower surface tension of HDD can be translated to better spreading and glide in formulations and position it comparably with mineral oils with similar viscosities and LEX FEEL 350.

Results of the measurements for 3 new compounds—HDD Glycotech non-redistilled original lot, RUDOL, and PROTOL-white mineral oils. HDD and HDD Glycotech have very close viscosity values by capillary viscosity but non-redistilled HDD has a slightly higher viscosity by rheology 61.5 vs. 68.2 respectively, which might be evidence of the presence of oiligomeric compounds. PROTOL viscosity is quite close to HDD being 63.3 by capillary and 68.2 by rheology therefore this mineral oil will be used in further formulations for HDD positioning.

The highest gloss value is observed for PROTOL and RUDOL mineral oils with both HDDs compared to them. ERVOL mineral oil and Squalane have a somewhat lower gloss value. Tested dimethicones have the lowest gloss. All these data are in good agreement with our and NPD sensory evaluations where shine, glide, and lubrication were appreciated for HDD (redistilled).

### Example 10: Cosmetics Containing Only Sugarcane Derived Compounds

Two cosmetic formulations were prepared using only ingredients derived from sugarcane through fermentation with host cells that were genetically engineered to contain the biochemical pathway to produce the ingredient, or a precursor ofthe ingredient that is then converted to the final ingredient using organic chemistry methods, or derived from sugarcane waste material. The ingredients generated and used include: squalane, hemisqualane, hexahydrofarnesyl-hexahydrofarnesoate, hexahydrofarnesyl ethanoate, hexahydrofarnesoic acid, hexahydrofarnesene dimer, hexahydrofamesol, carboxymethyl holocellulose, ectoine, cannabigerol (CBG), dihexahydrofarnesyl ascorbate, and patchoulol. The compositions of the two formulations and the function of each component is shown in

TABLE 2

| | | | Formula | | |
|---|---|---|---|---|---|
| Ingredient | Origin | Function | 1 | 2 | 3 |
| squalane | bio- | emollient | 6.5% | 6.5% | |
| | fermentation + | | | | |
| | chemistry | | | | |
| hemisqualane | bio- | sensory modifier, | 3.0% | 3.0% | |
| | fermentation + | organic solvent | | | |
| | chemistry | | | | |
| hexahydrofarnesyl- | bio- | emollient, | | | 2.5% |
| hexahydrofarnesoate | fermentation + | sensory modifier, | | | |
| | chemistry | organic solvent | | | |
| hexahydrofarnesyl | bio- | emollient, | 2.5% | 2.5% | |
| ethanoate | fermentation + | sensory modifier, | | | |
| | chemistry | organic solvent | | | |
| hexahydro-farnesoic | bio- | emulsifier | 3.5% | 3.5% | 3.5% |
| acid | fermentation + | | | | |
| | chemistry | | | | |
| hexahydrofarnesene | bio- | emollient, | | | 6.0% |
| dimer | fermentation + | sensory modifier | | | |
| | chemistry | | | | |
| hexahydro-farnesol | bio- | co-emulsifier, | | | |
| | fermentation + | emollient | | | |
| | chemistry | | | | |
| carboxymethyl | sugarcane waste | rheology | 1.9% | 3.1% | 2.0% |
| holocellulose | | modifier | | | |
| | | (thickener) | | | |
| ectoine | bio- | moisturizing | 1.0% | 1.0% | 1.0% |
| | fermentation | agent | | | |
| cannabigerol | bio- | anti- | 1.0% | 1.0% | 1.0% |
| (cbg) | fermentation | inflammatory, | | | |
| | | preservative | | | |
| di-hexahydrofarnesyl | bio- | anti-aging active, | | | |
| ascorbate | fermentation + | anti-oxidant | | | |
| | chemistry | | | | |
| patchoulol | bio- | fragrance | 0.025% | | |
| | fermentation | | | | |

### Example 11. Sensorial attributes of hexahydrofarnesyl-hexahydrofarnesoate (Compound (I-C)), ethyl 3,7,11-trimethyldodecanoate (Compound (I-A)), Isopropyl 3,7,11-trimethyldodecanoate (Isopropyl hexahydrofarnesoate, Compound (I-B))

General testing employed an analysis to compare and contrast certain sensorial attributes of ingredients, including analysis at rub out, t=0 (immediate afterfeel) and at t=5 minutes for afterfeel, and product manipulation.

Attributes tested were slip (test one), spreadability, cushion, gloss, and absorbency (test two), and after-feel (persistent emolliency) at 5 minutes (test three).

### Testing Procedures

General Skin Preparation: An expert washed the volar area of forearm prior to testing using a non-abrasive soap such as Ivory or Neutrogena. No other product was applied. No skin site was re-used.
Test One: Slip: Technician dispensed 0.1 cc of product to index or middle finger of expert. Panelist rubbed product between finger and thumb and scored the product as: Drag-very difficult, difficult, medium, easy, or very easy-Slip.
Test Two: Spreadability, Gloss, Cushion, and Asborbency (Rub-out): The expert used a skin scribe to define a circle 2″ in diameter on forearm. Product (0.1 cc) was dispensed into center of circle and a finger was used to spread the product in a circular motion. At three rubs, the product was evaluated for:
Spreadability: Least-very difficult, difficult, medium, easy, very easy-Most;
Gloss: Least-low, medium/low, medium, glossy, very glossy-Most;
Cushion: Least-low, medium/low, medium, cushiony, high cushion-Most; and
Absorbency: ranked absorption after 10 rubs as Least-low, medium/low, medium, good absorbency, high absorbency-Most
   Test Three: After-Feel (PersistentEmoliency) at 5 minutes: The expert ranked the product after 5 minutes for after-feel: Least-low, medium/low, medium, good, high-Most

The results are shown in Table 3A and Table 3B. The viscosity, gloss, surface tension, and wetting were also calculated for farnesyl HH-farnesoate (Compound (I-C)) and the results are shown in Table 4. The properties of Table 4 were determined as follows: Measurement of dynamic viscosity using rheometer: Using a ARES G2 rheometer, the viscosity of the samples were measured using 25 mm plate clamps with a gap size about 0.5 mm, strain 1%, frequency range 1-500 rad/s, and a temperature of about 25° C.

Measurement of surface tension of liquids: A Kruss DSA 100 was employed using the pendant drop method. Prior to the measurement, surface tension of the water was tested as a standard with an expected surface tension of 72 mN/m at 25° C. Then, a drop of the tested liquid was pushed through a 0.514 mm needle of a micro syringe and the surface tension was calculated using inbuilt software. The result of the measurement is an average of at least 3 measurements.
Static contact angle measurement (wetting): To measure the contact angle, a Kriss DSA 100 and the sessile drop method were used. A glass substrate was used for the measurements. To measure the contact angle on the substrate, a single drop was deposited onto the substrate from a 0.514 mm needle of a micro syringe. Then, drop shape and contact angle were analyzed using inbuilt software. The result of the measurement was the average of at least 3 measurements. To ensure that the substrate is prepared well, it was cleaned with non-polar hexane, THF, and polar acetone. To ensure that the glass substrate was clean, a drop of water was deposited on its surface.
Gloss measurement: To measure the gloss, 2 mL of material was drawn downed on a glass substrate. Then glass was placed over a black matte surface and gloss was measured with a BYK micro-TRI-gloss glossmeter.

Isopropyl hexahydrofarnesoate (Compound (I-B)) was not tested for further sensory evaluations due to its odor.

TABLE 3A
Farnesyl HH-farnesoate (Compound (I-C)) sensory evaluation

| Test | Rating |
|---|---|
| Spreadability | Easy |
| Slip | Easy |
| Gloss | Very |
| Cushion | High |
| Absorbency | Good |
| Emolliency | Good |

TABLE 3B
HH-farnesyl ethanoate (Compound (I-A)) sensory evaluation

| Test | Rating |
|---|---|
| Spreadability | Easy |
| Slip | Easy |
| Gloss | Very |
| Cushion | High |
| Absorbency | Good |
| Emolliency | Medium |

TABLE 4
Additional parameters of the Farnesyl HH-farnesoate
(Compound (I-C)) sensory evaluation

| Test | Rating |
|---|---|
| Viscosity | 30 ± 10 cP at 25° C. |
| Gloss | 69.4 ± 2.7 |
| Surface tension | 27.6 ± 0.2 mN/m |
| Wetting | 18.5° |

As shown in Tables 3A and 3B, both farnesyl HH-farnesoate (Compound (I-C)) and HH-farnesyl ethanoate (Compound (I-A)) were very glossy, which helps to make the compounds visually appealing, particularly shiny. Spreadability and slip are related to the surface tension and viscosity of compositions; low surface tension results in compositions that easily spread and are slippery, characteristics typically related to the high foaming and lathering ability often found in face washes and hand soaps. Further, compositions with high viscosity often spread easily and smoothly while feeling soft on the skin, properties of hand creams. Both compounds also exhibited easy slippage and spreadability and farnesyl HH-farnesoate (Compound I-C) exhibited a surface tension of 27.6±0.2 mN/m and a viscosity of 30±10 cP at 25° C. Compounds that feel slippery also improve the feel on skin and help to detangle hair.

### Example 12. Skin Permeation and Delivery Measurements of Compounds (TI-A), (II-B), and (II-C)-Containing Formulations

The rate and extent of in vitro skin permeation of compounds tetrahexydecyl ascorbate (THDA, shown below), di(hexahydrofarnesyl) ascorbate (Compound (II-A)), tri(hexahydrofarnesyl) ascorbate (Compound (II-B)), and tetra(hexahydrofamesyl) ascorbate (Compound (II-C)) was measured into and through cadaver skin using a Franz diffusion cell system. THDA is a Vitamin C ester commonly used in cosmetic formulations.

Flux was measured over a period of 24 hours after application of the formulations. At the end of the 24 hour study, the skin was washed, tapestripped and split into epidermal and dermal compartments. The compounds were then extracted from the skin compartments and the compound concentration was measured in these compartments along with the receptor fluid aliquots. The formulations were chosen to determine the effect of the formulation composition and compound concentration on the rate of delivery of the compounds into and through the skin. In vitro skin permeation measurements with Franz-type diffusion cells provide data that is informative relative to in vivo performance.

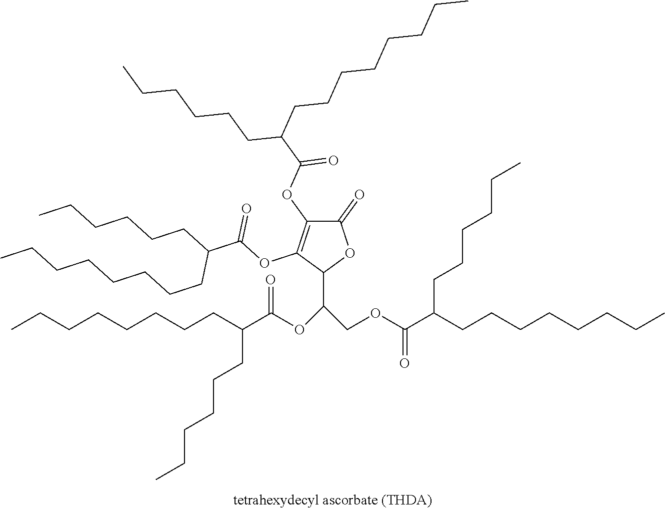 [SMILES: CCCCCCCCC(CCCCCC)C(=O)OCC(OC(=O)C(CCCCCC)CCCCCCCC)C1OC(=O)C(OC(=O)C(CCCCCC)CCCCCCCC)=C1OC(=O)C(CCCCCC)CCCCCCCC]

### Formulation Details

Details of the formulations are listed in Table 5 below. To prepare the formulations, the compounds were weighed in a glass vial into which the squalane was added. The ending solution was then vortexed for 1 minute, followed by sonication for 30 minutes.

TABLE 5
Formulations of Compounds in Study

| | F005 | F006 | F007 | F008 |
|---|---|---|---|---|
| Ingredients | wt/wt % | wt/wt % | wt/wt % | wt/wt % |
| Tetrahexydecyl ascorbate | 1.00 | | | |
| Di(hexahydrofarnesyl) | | 1.00 | | |
| ascorbate (Compound (II-A)) | | | | |
| Tetra(hexahydrofarnesyl) | | | 1.00 | |
| ascorbate (Compound (II-B)) | | | | |
| Tri(hexahydrofarnesyl) | | | | 1.00 |
| ascorbate (Compound (II-C)) | | | | |
| Squalane | 99.0 | 99.0 | 99.0 | 99.0 |

### Skin Sourcing and Equipment

Intact human cadaver skin was purchased from Science Care. The skin tissue was dermatomed by the tissue bank to a thickness of some 250-500 μm and shipped frozen on dry ice. Upon receipt of the donor skin, the skin pieces were stored at −20° C. until used. Prior to use, the skin pieces were removed from the freezer and allowed to thaw fully at ambient temperature.

The following equipment was used during the course of the study:
1.) Diffusion cells. 24 diffusion cells with 3.3 ml receptor volume and a 0.5 5 cm² receptor fluid exposure surface area.
2.) Stirring Dry Block Heaters. Reacti-Therm #18823 stirring dry block heaters were used to maintain the receptor fluid at 32±0.5° C. with constant stirring throughout the study.
3.) The analysis was carried out with an Agilent 1260 HPLC with a G6120 MS detector and a G4212B diode array detector.

### Analytical Methods

A liquid chromatography mass spectrometry (“LC-MS”) and HPLC analytical method was implemented for the detection of the compounds.

### Preparation of Mobile Phases

Mobile Phase A: Mobile Phase A was prepared by measuring 1000 ml of LC-MS grade water in a volumetric cylinder and then transferring the contents into a 2 L glass media bottle. Formic acid (1 ml) was then pipetted into the media bottle. Ammonia formate (10 mM) was then weighed using an analytical balance and added to the media bottle. The resulting mixture in the media bottle was shaken until the contents were fully mixed and the ammonia formate dissolved.

Mobile Phase A was stored for less than one week during the course of the analysis.

Mobile Phase B: Mobile Phase B was prepared by measuring 1000 ml of LC-MS grade methanol in a volumetric cylinder and then transferring the contents into a 2 L glass media bottle. Formic acid (1 ml) was then pipetted into the media bottle. Mobile Phase B was stored for less than one week during the course of the analysis

Mobile Phase C: Mobile Phase C was prepared by measuring 1000 ml of LC-MS grade isopropanol in a volumetric cylinder and then transferring the contents into a 2 L glass media bottle. Mobile Phase C was stored for less than one week during the course of the analysis.

### Preparation of Stock Solution and Calibration Standards

Individual calibration standards were prepared for each of the compounds. A compound stock solution was first prepared by weighing 4 mg of the compound with an analytical balance in a glass vial. The vial was then tared on the balance and 4 ml of 50 vol %/50 vol % methanol/isopronaol with 0.1 vol % formic acid was introduced into the glass vial with a pipettor. The vial was reweighed. The vial was then removed from the analytical balance and capped. The capped vial was vortexed and sonicated using an ultrasonication bath until the compound was fully dissolved.

The above procedure was used to make a 1 mg/ml Stock Solution for the compound. Further calibration standards were prepared through serial dilution. In each serial dilution, 300 μl of the preceding calibration standard was diluted with 1200 μl of 50 vol %/50 vol % methanol/isopronaol with 0.1 vol % formic acid. The compound concentration in each of the calibration standards is shown in Table 6 below.

TABLE 6
Calibration standards and the corresponding
concentration of the compounds

| | Active | |
|---|---|---|
| | Vitamin C derivative | |
| | Calibration standard | |
| | Conc (mg/ml) | |
| | Stock Solution | |
| | 1000 μg/ml Stock Solution | |
| Cal 2 | 200 | μg/ml |
| Cal 3 | 40 | μg/ml |
| Cal 4 | 8 | μg/ml |
| Cal 5 | 1.6 | μg/ml |
| Cal 6 | 0.32 | μg/ml |
| Cal 7 | 0.064 | μg/ml |
| Cal 8 | 0.0128 | μg/ml |

The compound was first prepared in a 1 mg/ml stock solution. Calibration standards were then serially diluted five-fold with 50 vol %/50 vol % methanol/isopropanol with 0.1 vol % formic acid from the stock solution. Cal3-Cal8 was used for the calibration curve. The same protocol was separately carried out for each compound. The study samples were collected during the permeation study with no further preparation performed on the samples prior to analysis.

### Chromatographic Parameters

An outline of the analytical method details is provided in Table 7 (for di(hexahydrofarnesyl) ascorbate (Compound II-A) and tri(hexahydrofarnesyl) ascorbate (Compound II-B)) and Table 8 (for tetra(hexahydrofarnesyl) ascorbate (Compound 11-C) and tetrahexydecyl ascorbate) below.

TABLE 7
Chromatographic parameters for the detection of di(hexahydrofarnesyl)
ascorbate (Compound (II-A)) and tri(hexahydrofarnesyl)
ascorbate (Compound (II-B))

| Instrument: | Agilent 1260 HPLC/UV/MS | | |
|---|---|---|---|
| Column: | Poroshell 120 EC-18 2.1 × 50 mm, 4 mm | | |
| Mobile phase: | A: 0.1% Formic acid in water with | | |
| | 10 mM Ammonia Formate | | |
| | B: Methanol with 0.1% Formic acid | | |
| | Time (minutes) | % A | % B |
| Gradient: | 0 | 10 | 90 |
| | 1 | 10 | 90 |
| | 1.5 | 4 | 96 |
| | 10 | 4 | 96 |

| | |
|---|---|
| Flow rate: | 0.5 ml/min |
| Column temperature: | 40° C. |
| MS | MS SIM Negative: 623.4 (M − H) |
| Di(hexahydrofarnesyl) | |
| ascorbate: | |
| MS | MS SIM Negative: 847.6 (M − H) |
| Tri(hexahydrofarnesyl) | |
| ascorbate: | |
| Injection volume: | 5 μl |

TABLE 8
Chromatographic parameters for the detection of tetra(hexahydrofarnesyl)
ascorbate (Compound (II-C)) and tetrahexydecyl ascorbate

| Instrument: | Agilent 1260 HPLC/UV/MS | | | |
|---|---|---|---|---|
| Column: | Poroshell 120 EC-18 2.1 × 50 mm, 4 mm | | | |
| Mobile phase: | A: 0.1% Formic acid in water with | | | |
| | 10 mM Ammonia Formate | | | |
| | B: Methanol with 0.1% Formic acid | | | |
| | C: Isopropanol | | | |
| | Time (minutes) | % A | % B | % C |
| Gradient: | 0 | 5 | 75 | 20 |
| | 2 | 5 | 25 | 70 |
| | 9 | 5 | 25 | 70 |

| | |
|---|---|
| Flow rate: | 0.5 ml/min |
| Column temperature: | 40° C. |
| MS | MS SIM Positive: 1090.8 (M + NH4) |
| Tetro(hexahydrofarnesyl) | |
| ascorbate: | |
| MS Tetrahexydecyl | MS SIM Positive: 1146.9 (M + NH4) |
| ascorbate: | |
| Injection volume: | 5 μl |

### Calculation

After the analytical testing was complete, the samples were analyzed using Chemstation software. The areas under the curve (“AUCs”) of the compound peaks were recorded and converted to jig/ml values using a calibration curve developed from the calibration standards' AUC values and known concentration values. The concentrations were then multiplied by the receptor volume (3.3 mL) and divided by the surface area of the skin exposed to the receptor fluid (0.55 cm²) for an end cumulative amount in μg/cm².

For receptor fluid time points greater than 4 hrs, this μg/cm² value was corrected for the sample aliquot volumes which were removed to compensate for the dilution caused by replacing the sample volume with fresh buffer solution. As an example, for the second time point at 8 hrs, the dilution factor (300 μl aliquot/3.3 ml receptor volume or 1/11) is multiplied by the g/cm² value calculated for the 4 hr time point, the result of which is then added to the μg/cm² concentration which is calculated using the 8 hr AUC value.

### Receptor Fluid

The receptor fluid consisted of phosphate buffered saline at pH 7.4, sourced from Quality Biologicals, with 0.01 wt % NaN₃ (added as a preservative), and lwt % Brij 020 (added as a solubilizing agent). The solubility of the compounds in the receptor fluid was determined to be sufficient to maintain sink conditions throughout the study. After mixing the receptor fluid, the receptor fluid was degassed, filtered through a ZapCap CR 0.2 μm membrane under vacuum, and stirred for an additional 20 minutes under vacuum.

### Experimental Procedure

### Skin Preparation

Human cadaver skin from Science Care was prepared by removing the cadaver skin piece from the package and placing in a distilled water bath for 10 minutes to defrost the skin. The skin was then unfolded and floated on the surface of the water bath and examined for defects. The skin was then removed from the water bath and placed in a beaker with 250 ml of distilled water where it was allowed to sit in the water bath for 5 minutes. This process was repeated a second time to ensure the cryoprotectant was thoroughly washed away from the skin. The skin was then removed from the beaker and placed on aluminum foil. The exterior surface of the skin was then patted dry with a KimWipe.

### Assembling the Franz-type Diffusion Cells

Glass FDCs with a 3.3 ml receiver volume and 0.55 cm² diffusional area were used. Once the skin was defrosted and washed as described above the FDCs were prepared as follows:
1. The receptor wells were filled with degassed receptor fluid using a pipette.
2. A 6 mm by 3 mm diameter Teflon coated magnetic stir bar was introduced into each receptor well.
3. The defrosted and washed cadaver skin pieces were examined and only areas of even thickness and with no visible surface damage were used.
4. The skin pieces were cut into approximately 2 cm×2 cm squares using a razor blade. The square sizes were adjusted as necessary according to the shape and dimensions of the skin piece but were selected to be approximately uniform in size among all FDCs.
5. A skin piece was centered on each inverted donor compartment, with the stratum corneum (“SC”) side contacting the donor compartment.
6. The donor and receptor well compartments were then aligned and clamped together with a pinch clamp, ensuring that the skin pieces were centered between both donor
and receptor wells.
7. Additional receptor fluid was added as necessary. Air bubbles in the receptor well, if any, were removed by tilting the FDC assembly such that the air escaped along the sample port. Receptor wells were filled with approximately 3.3 ml of receptor fluid.
8. The assembled FDCs were placed into stirring dry block heaters which were preheated to 32° C. The receptor fluid was continuously agitated via the magnetic stir bar.
9. After 20 minutes, the surface of the skin in each FDC was examined. If the skin appeared wet or showed signs of sweating, the cell was discarded.

### Membrane Integrity Check

Once the FDCs were assembled, the barrier integrity of the skin pieces was tested prior to application of the test articles by a transepithelial electrical resistance reading (“TEER”):
1. An aliquot of 150 μl of PBS was introduced into each FDC donor well. The buffer salts allow for electrical conductivity.
2. After 10 minutes, a blunt electrode probe was placed into the donor well. The probe rests lightly on the surface of the skin under its own weight.
3. A second electrode was then inserted into receptor fluid via the sample port on the receptor chamber of the FDC.
4. An alternating current (“AC”) signal, 100 mV root mean square (“RMS”) at 100 Hz, was applied across the skin using a waveform generator. The impedance was then measured with a digital multimeter and the results recorded in kΩ.
5. After impedance analysis was complete, results were analyzed. Any FDC showing anomalously low impedance (nominally <2 kΩ), were discarded.
6. The FDCs were ranked according to the magnitudes of the measured impedance readings. Test articles were then assigned to the batch of FDCs such that the replicates for each test article are each applied to a skin piece with nearly equivalent average impedance values. The ranking of skin pieces was carried out separately for each donor skin piece.
7. The PBS was removed from each FDC donor cell. The surface of the skin was tapped dry with a Kimwipe.
8. The entire volume of Receptor Fluid was removed from each FDC and replaced with fresh Receptor Fluid.
9. The FDCs were finally placed into preheated dry block heaters.

### Formulation Application and Results

After the membrane integrity tests were complete and the cells appropriately sorted, samples of the formulations were applied to the stratum corneum of the skin. A one-time dosing regimen was used for this study. The approximate dose of the compound applied per cell and the corresponding formulation is shown in Table 9.

TABLE 9
Compound dose per cell for the applied formulations

| | | | | | Nominal dose |
|---|---|---|---|---|---|
| Study | Test | | | | Active per |
| Arm | Articles | Dosing | Active | % Active | cell in μg/cm² |
| 1 | F005 | 5 μl | Tetrahexydecyl ascorbate | 1.0 wt % | 90.91 μg/cm² |
| 2 | F006 | 5 μl | Di(hexahydrofarnesyl) | 1.0 wt % | 90.91 μg/cm² |
| | | | ascorbate (Compound (II-A)) | | |
| 3 | F007 | 5 μl | Tri(hexahydrofarnesyl) | 1.0 wt % | 90.91 μg/cm² |
| | | | ascorbate (Compound (II-B)) | | |
| 4 | F008 | 5 μl | Tetra(hexahydrofarnesyl) | 1.0 wt % | 90.91 μg/cm² |
| | | | ascorbate (Compound (II-C)) | | |

The nominal dose assumes a specific gravity of 1.0 for the formulation and assumes 100% of the applied 5 μl of the formulation remains on the skin after spreading the formulation across the skin surface. For the dosing procedure, a 5 μl dose of the formulation was applied to the skin using a positive displacement Nichiryo pipettor. The 5 μl dose was then spread across the surface of the skin using the blunt end of a glass rod.

Using a graduated Hamilton type injector syringe, a 300 μl aliquot was abstracted from the sampling port of each FDC at each of 4, 8 and 24 hours. Fresh receptor fluid was added to each receptor well to replace the volume of fluid abstracted. Each abstracted aliquot was introduced into a well in a 96-well microtiter plate. Samples were stored in a refrigerator at 4-8° C., protected from light, prior to LC-MS analysis. Samples were analyzed within 10 days of collection.

At 24 hours, the surface of the skin was wiped and washed twice using 200 μl of a 50 vol %/50 vol % water/ethanol mixture. The skin was then tapped dry using KimWipes.

After the washing step was complete, the skin was then tapped dry and tapestripped three times with cellophane tape, each tapestripping consisting of applying a piece of cellophane tape to the skin, pressing the tape piece onto the skin and then peeling off the tape, thereby systematically removing the upper most layers of the stratum corneum. The tapestripping was done three times. All three tape strips were discarded.

After the tapestripping was complete, the remaining skin was split into epidermal and dermal compartments by using a pair of spatulas. The epidermal and dermal compartments were then separately placed into glass vials, into which 3 ml of 50 vol %/50 vol % methanol/isopronaol with 0.1 vol % formic acid was added. The skin pieces were then incubated at 40° C. for 24 hours with gentle agitation. After the 24 hour incubation period, sample aliquots were collected.

The samples were abstracted from the receptor wells, and the epidermal or dermal layers were analyzed using the LC-MS and HPLC methods described above. The concentrations of the compounds were assayed and reported in each case.

The accumulated dose of the compounds at each of the time points is shown in Table 10. The measurement at 4, 8, and 24 hours is the amount of compound that that passed through the skin into the reservoir. The amount of compound that passed through to the epidermis and dermis was measured at 24 hours.

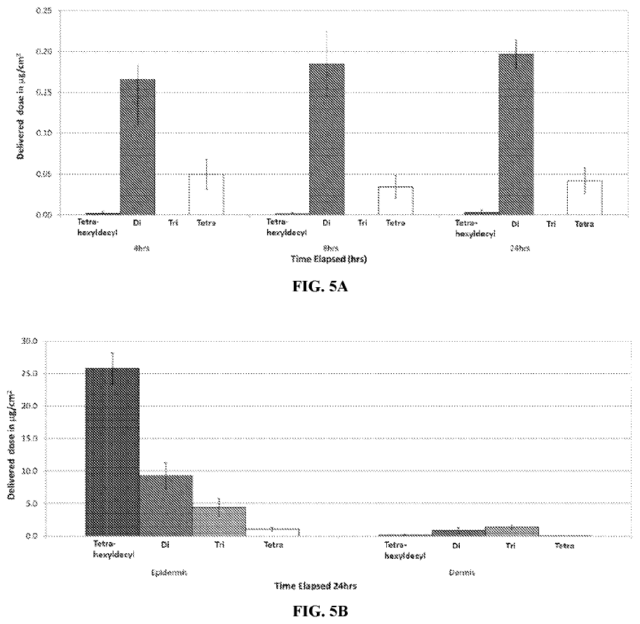

FIG. 5A is a graph of the amount of each compound that that passed through the skin into the reservoir at each of the time points. FIG. 5B is a graph of amount of compound that accumulated in the dermis and epidermis at the 24 hour time point. As shown in Table 10 and FIG. 5B, di(hexahydrofarnesyl) ascorbate (Compound (II-A)) and tri(hexahydrofarnesyl) ascorbate (Compound (II-B)) showed better penetration to the dermis, where collagen is found, than THDA, a Vitamin C derivative commonly used in cosmetic applications.

TABLE 10
Total accumulated dose (in μg/cm²) of the
compounds delivered into and through the skin.

| | Tetrahexy- | Di(hexahydro- | Tri(hexahydro- | Tetra(hexahydro- |
|---|---|---|---|---|
| | decyl | farnesyl) ascorbate | farnesyl) ascorbate | farnesyl) ascorbate |
| Time (hrs) | ascorbate | (Compd (II-A)) | (Compd (II-B)) | (Compd (II-C)) |
| 4^{a} | 0.00 | 0.17 | 0.00 | 0.05 |
| 8^{a} | 0.00 | 0.18 | 0.00 | 0.03 |
| 24^{a} | 0.00 | 0.20 | 0.00 | 0.04 |
| Epidermis^{b} | 25.78 | 9.28 | 4.41 | 1.02 |
| Dermis^{b} | 0.18 | 0.85 | 1.37 | 0.01 |
| Time (hrs) | StdErr | StdErr | StdErr | StdErr |
| 4^{a} | 0.00 | 0.06 | 0.00 | 0.02 |
| 8^{a} | 0.00 | 0.04 | 0.00 | 0.01 |
| 24^{a} | 0.00 | 0.02 | 0.00 | 0.02 |
| Epidermis^{b} | 2.39 | 2.02 | 1.37 | 0.24 |
| Dermis^{b} | 0.06 | 0.43 | 0.30 | 0.01 |

^{a}At 4, 8, and 24, the amount of compound that passed through the skin into the reservoir was determined
^{b}Meaured at 24 hours

### Example 13. Sustainable and scalable synthesis of hexahydrofarnesoic acid (HHFA) Materials and Methods

Hexahydrofarmesol (HIIF) was generated by the hydrogenation of farnesol as described previously in U.S. Pat. No. 8,519,204. Sodium hypochlorite (NaOCl, 500 or 120%), sodium chlorite, (NaClO₂, 80-85%), TEMPO (98%), mono-sodium hydrogen phosphate hydrate (NaH₂PO₄·H₂O), sodium thiosulfate pentahydrate (Na₂S₂O₃·5H₂O; ACS grade) and ethyl acetate (ACS grade) were purchased from commercial sources and used directly without further purification. ¹H and ¹³C NMR spectra were recorded at 500 and 125 MHz, respectively. Spectral values are reported in ppm (δ), and coupling constants, J in Hertz. Samples were analyzed by GC-MS (squalene 22 method) and values are reported as area 00. Weight/weight percent assay developed by PAC (HHF-HIIFA-GC-FfD-SOP01326) was used for quantification of hexahydrofarnesoic acid.

### Optimization of the Synthesis of HHFA

Following the protocol described in Zhao, M. M. et al., Organic Syntheses Coll. Vol., 2005, 81, 195-203; 2009, 11, 107-113 and Zhao, M. M. et al., J. Org. Chem. 1999, 64, 2564-2566, HHF was heated with TEMPO, bleach, and sodium chlorite. Oxidation proceeded smoothly within an hour to form HHFA in 97% area purity.

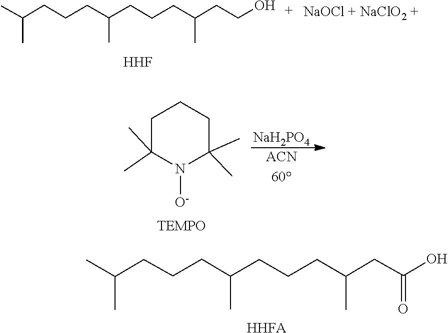

Parameters of the reaction were then optimized to develop a more sustainable and cost-efficient process. First, ethyl acetate and acetone were tested as alternatives to acetonitrile as a solvent. Under the same conditions described above using acetonitrile, when acetone was used a solvent, GC-MS of the reaction showed an area of only 55% for HHFA and an area of 28% for unconverted HHF. The use of ethyl acetate led to the formation of HHFA in 96% area by GC-MS and no unconverted HHF.

Next, the molar ratio of TEMPO and NaH₂PO₄ were reduced by 50% and 75% respectively, and the solvent usage by 75%. Oxidation proceeded smoothly at room temperature within an hour with no residual HHF or the aldehyde intermediate. To increase the product output per batch, concentrated solutions of bleach (12%) and NaH₂PO₄ (2.93 M) were tested. A concentrated solution of bleach is traditionally associated with increased side reactions due to higher hypochlorite (Ocl-) concentration, hypochlorous acid (HOCl) and available chlorine (Cl₂), but lowering the temperature from 60° C. to room temperature (20° C.) allowed the use of concentrated bleach while maintaining a crude purity of 97%.

The oxidation reaction was next run in the absence of solvent. HHF was treated with TEMPO, bleach, and sodium chlorite at different reaction scales at room temperature. In the absence of solvent, GC-MS analyses showed a significant drop in crude purity of 15-30%. The major product was the acid anhydride formed by the condensation of two molecules of HHFA:

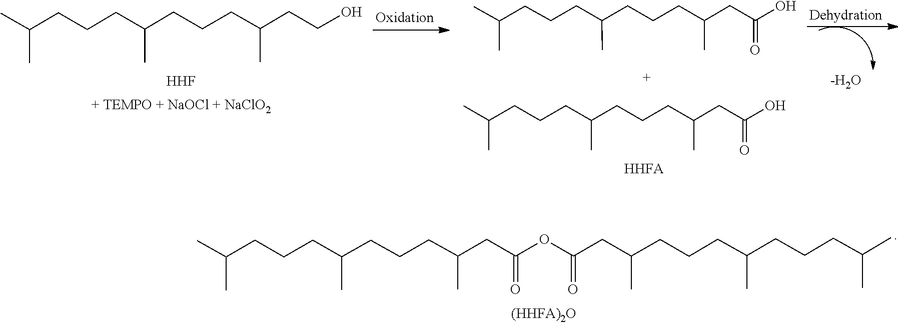

Since TEMPO oxidations are strongly exothermic and a temperature jump of 50° C. was observed, better temperature control strategies were investigated, including lowering the reaction temperature to potentially decrease the formation of the acid anhydride. Additionally, mixtures of bleach and sodium chlorite are exothermic, so diluted solutions of co-oxidants were simultaneously added instead. Subsequently, to maximize product output, concentration of bleach, and sodium chlorite were increased because the exotherm could be controlled.

A solution of HHF, TEMPO, and sodium phosphate were cooled to 0-5° C., followed by the simultaneous addition of 5% bleach and 2.5M of NaClO₂. Lowering the temperature significantly reduced the formation of the anhydride, restoring the crude purity back to >95%. To simplify the process, the hypochlorite (Ocl⁻) was added in a single portion and the concentrated sodium chlorite (NaClO₂) was dosed into the reaction vessel. Several batches were produced using this process to afford an average isolated yield of 87% and 93% weight purity. Doubling the concentration of the oxidants afforded crude HHFA at 95 area % by GC-MS, despite a temperature jump of 50° C. In subsequent runs, better temperature control was achieved using jacketed reactors. Distillation using a Kugelrohr short-path unit separated the relatively volatile fane impurities at 110-145° C. and the product was distilled at 155-165° C., at 0.2-0.35 torr. Overall running the reaction in the absence of solvent enabled loading twice the amount of the starting material for the same reaction volume, affording twice the amount of product. Thus, the product output per batch improved by 2× from 142 grams of HHFA/L of the reaction mixture to 293 grams HHFA/L.

To enable better temperature control and efficient mixing, reactions on a 100-300 g scale were conducted in a jacketed reactor. HHF (100 g) was oxidized in a 300 mL reactor, filling the reactor capacity to >90% and isolation of this material afforded HHFA in 84% yield and 94.8 weight % purity. While scaling the reaction on a 300 g scale, a 50% reduction in time was realized by reducing the amount of NaClO₂ to a single equivalent with a higher concentration (4.93M) to provide a yield of 94% and weight purity of 92%.

### Detailed Procedure of the Optimized Process for the Synthesis of HHFA

A 5-necked ChemGlass 3 L batch reactor was fitted with (i) an overhead stirrer; (ii) a thermocouple; (iii) a syringe pump to add NaClO₂; (iv) a trap containing 2.5M Na₂S₂O₃ to quench chlorine; and (v) an outlet open to air. The reaction flask was cooled to 0-5° C., followed by the addition of HHF (300 g, 1.313 mol), ~3M NaH₂PO₄ (185 mL, 0.41 equiv.), TEMPO (10.2 g, 0.072 mol, 0.05 equiv.) and 12% Bleach (84 mL, 0.106 equiv.). Using a syringe pump, 4.93M NaClO₂ (183 mL, 1.0 equiv.) was added over a period 5 hours at rates shown below. The total reaction volume was 1 L. The reaction temperature was monitored as shown below.

| | Rate | | | Temp. |
|---|---|---|---|---|
| Step | (mL/min) | Initial ° C. | Final ° C. | Jump |
| Addition of NaClO₂ | 0.7 | 5 | 7.2 | 2.2 |
| | 0.8 | 7.2 | 9.2 | 2 |
| | 0.9 | 9.2 | 12 | 2.8 |
| Addition of NaClO₂ | 1 | 12.7 | 15 | 2.3 |
| | 1.2 | 16.5 | 24 | 7.5 |

The reaction was monitored by GC-area % for the disappearance of starting material, which happened within 1 hour after the completion of NaClO₂ addition. The reaction was quenched by the addition of solid Na₂S₂O₃ at 5° C. The pH of the reaction mixture was acidified with 4M H₂SO₄ to convert the sodium salt to the carboxylic acid to minimize loss during extraction. Ethyl acetate (1000 mL) of was added to the reactor, and the reaction mixture was transferred to a separatory funnel. The aqueous layer was extracted with ethyl acetate (500 mL×3); total organic layer was 2.5 L. The combined organic layer was dried (Na₂SO₄) and concentrated to give a crude mass of 350 g.

Kugelrohr Distillation: TEMPO and fane enriched samples were isolated from 120-145° C. at 0.9-0.385 Torr. Pure HHFA material (as a mixture of diastereomers) was isolated from 150-164° C. at 0.35 Torr. Yield, 94%, GC-AUC 99%; weight % purity is 92.1%.

¹H NMR (500 MHz, CDCl₃): δ 2.35 (ddd, J=15.0, 5.9, 2.7 Hz, 1H); 2.14 (ddd, J=15.0, 8.2, 2.7 Hz, 1H), 2.08-1.79 (m, 1H), 1.51 (dq, J=13.2, 6.6 Hz, 1H), 1.45-0.99 (cluster of multiplets, 13H), 0.97 (dd, J=6.7, 1.0 Hz, 3H), 0.86 (d, J=5 Hz, 6H), 0.86 (d, J=5 Hz, 3H), COOH not detected.

¹H NMR (500 MHz, DMSO): δ 11.96 (s, 1H), 2.18 (ddd, J=14.9, 6.0, 2.6 Hz, 1H), 1.99 (ddd, J=14.9, 8.0, 2.6 Hz, 1H), 1.81 (dq, J=13.2, 6.7 Hz, 1H), 1.50 (dq, J=13.2, 6.6 Hz, 1H), 1.41-0.97 (m, 12H), 0.85 (m, cluster of doublets, 12H,).

¹³C NMR (125 MHz, CDCl₃,) δ 180.38; 41.79 & 41.72; 39.42; 37.40, 37.31, 37.25, 37.19, 37.15, 37.05; 32.80 & 32.77; 30.20 & 30.18; 28.02; 24.86; 24.39; 22.61, 22.51; 19.62 19.58, 19.55, 19.51.

What is claimed:

1. A compound of Formula (II): 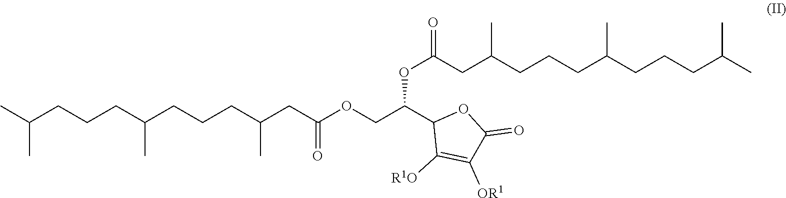
wherein R¹ is independently, at each occurrence, selected from H and 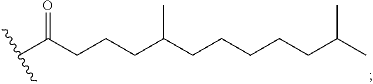 [SMILES: *C(=O)CCCC(C)CCCCCC(C)C]
or a salt and/or stereoisomer thereof.

2. The compound of claim 1, wherein the compound is Compound (II-A): 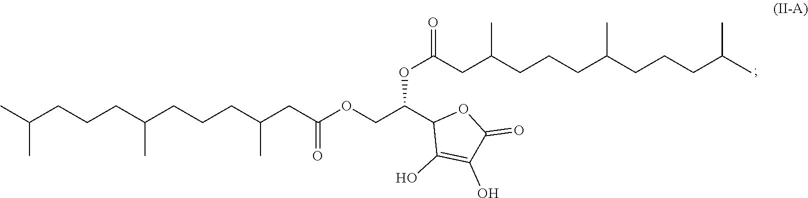 [SMILES: CC(C)CCCC(C)CCCC(C)CC(=O)OC[C@H](OC(=O)CC(C)CCCC(C)CCCC(C)C)C1OC(=O)C(O)=C1O]
or a salt and/or stereoisomer thereof.

3. The compound of claim 1, wherein the compound is Compound (II-B): 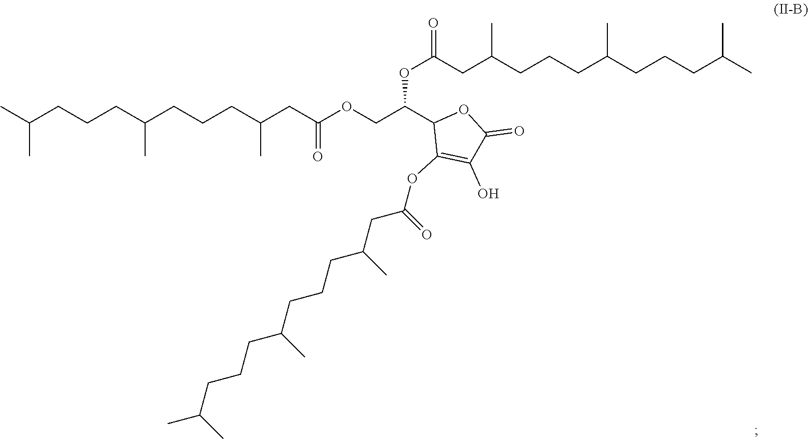 [SMILES: CC(C)CCCC(C)CCCC(C)CC(=O)OC[C@H](OC(=O)CC(C)CCCC(C)CCCC(C)C)C1OC(=O)C(O)=C1OC(=O)CC(C)CCCC(C)CCCC(C)C]
or a salt and/or stereoisomer thereof.

4. The compound of claim 1, wherein the compound is Compound (II-C): 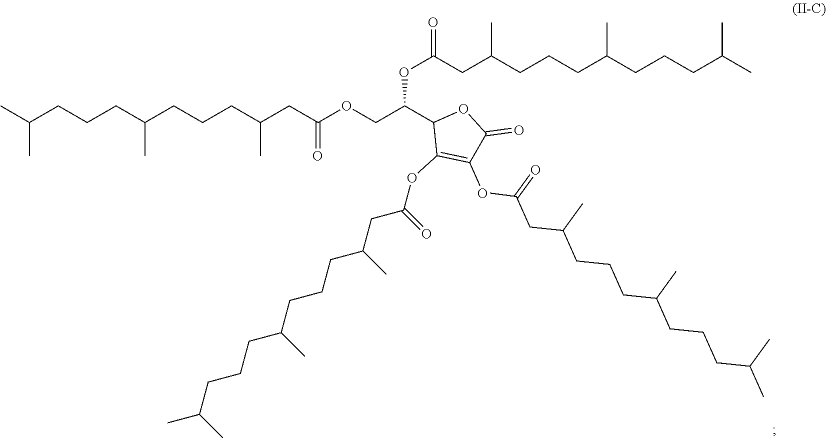 [SMILES: CC(C)CCCC(C)CCCC(C)CC(=O)OC[C@H](OC(=O)CC(C)CCCC(C)CCCC(C)C)C1OC(=O)C(OC(=O)CC(C)CCCC(C)CCCC(C)C)=C1OC(=O)CC(C)CCCC(C)CCCC(C)C]
or a salt and/or stereoisomer thereof.

5. Compound (I-C): 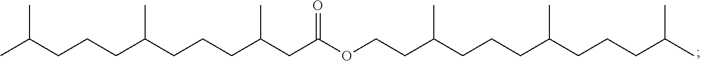 [SMILES: CC(C)CCCC(C)CCCC(C)CCOC(=O)CC(C)CCCC(C)CCCC(C)C]
or a salt and/or stereoisomer thereof.

6. A method of producing a compound of Formula (I): 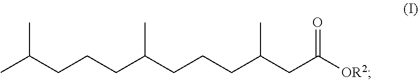
or a salt and/or stereoisomer thereof comprising contacting hexahydrofarnesoic acid: 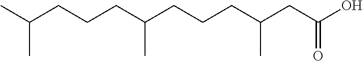 [SMILES: CC(C)CCCC(C)CCCC(C)CC(=O)O]
with R²OH and an acid; wherein R² is selected from a straight or branched C₁₋₆alkyl.

7. The method of claim 6, comprising contacting hexahydrofamesoic acid with ethanol to produce ethyl-hexahydrofarnesoate: 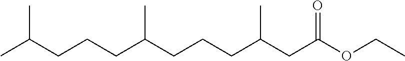 [SMILES: CCOC(=O)CC(C)CCCC(C)CCCC(C)C]
(Compound (I-A)).

8. The method of claim 7, wherein the acid is selected from hydrochloric acid, nitric acid, and sulfuric acid.

9. The method of claim 7 or 8, wherein the acid is sulfuric acid.

10. The method of claim 6, comprising contacting hexahydrofarnesoic acid with isopropanol to produce isopropyl-hexahydrofarnesoate: 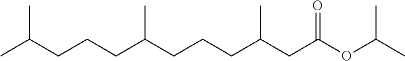 [SMILES: CC(C)CCCC(C)CCCC(C)CC(=O)OC(C)C]
(Compound (I-B)).

11. The method of claim 10, wherein the acid is selected from hydrochloric acid, nitric acid, and sulfuric acid.

12. The method of claim 10 or 11, wherein the acid is sulfuric acid.

13. A method of producing a compound of Formula (I): 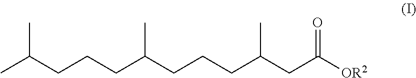
or a salt and/or stereoisomer thereof comprising:
converting hexahydrofarnesoic acid: 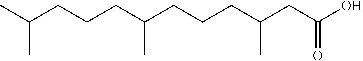 [SMILES: CC(C)CCCC(C)CCCC(C)CC(=O)O]
to hexahydrofarnesyl chloride: 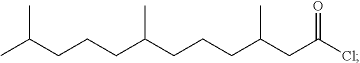 [SMILES: CC(C)CCCC(C)CCCC(C)CC(=O)Cl]
isolating the hexahydrofarnesyl chloride; and
contacting the hexahydrofarnesyl chloride with R²OH; wherein R² is selected from a straight or branched C₁₋₆alkyl.

14. The method of claim 13, to produce isopropyl-hexahydrofarnesoate: 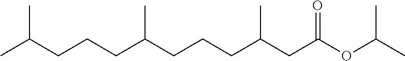 [SMILES: CC(C)CCCC(C)CCCC(C)CC(=O)OC(C)C]
(Compound (I-B)) comprising:
contacting hexahydrofarnesoic acid with dichloromethane, dimethylformamide, and thionyl chloride to form a reaction mixture;
concentrating the reaction mixture under vacuum; and
adding the concentrated reaction mixture to isopropanol.

15. A method of producing Compound (I-C) of claim 5, comprising:
contacting hexahydrofarnesoic acid: 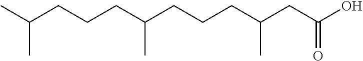 [SMILES: CC(C)CCCC(C)CCCC(C)CC(=O)O]
with hexahydrofarnesol: 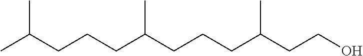 [SMILES: CC(C)CCCC(C)CCCC(C)CCO]
and a coupling agent.

16. The method of claim 15, wherein the coupling agent is selected from N-(3-dimethylaminopropyl)-N′-ethylcarbodiimide hydrochloride, N,N′-diisopropylcarbodiimide, and N,N′-dicyclohexylcarbodiimide.

17. The method of claim 15 or 16, wherein the coupling agent is N-(3-dimethylaminopropyl)-N′-ethylcarbodiimide hydrochloride.

18. The method of any one of claims 15-17, wherein the method further comprises contacting hexahydrofarnesoic acid with hexahydrofarnesol in the presence of 4-dimethylaminopyridine.

19. A method of producing Compound (I-C) of claim 5, comprising:
converting hexahydrofarnesoic acid: 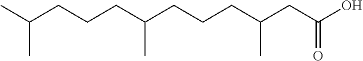 [SMILES: CC(C)CCCC(C)CCCC(C)CC(=O)O]
to hexahydrofarnesyl chloride: 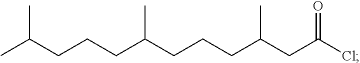 [SMILES: CC(C)CCCC(C)CCCC(C)CC(=O)Cl]
isolating the hexahydrofarnesyl chloride; and
adding the hexahydrofarnesyl chloride to hexahydrofarnesol:

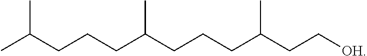 [SMILES: CC(C)CCCC(C)CCCC(C)CCO]

20. The method of claim 19, comprising:
contacting hexahydrofarnesoic acid with dichloromethane, dimethylformamide, and thionyl chloride to form a reaction mixture;
extracting hexahydrofarnesyl chloride from the reaction mixture; and
adding the hexahydrofarnesyl chloride to hexahydrofarnesol.

21. A method of producing a compound of any one of claims I-4, comprising:
contacting hexahydrofarnesoic acid: 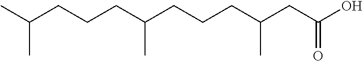 [SMILES: CC(C)CCCC(C)CCCC(C)CC(=O)O]
with ascorbic acid and a coupling agent.

22. The method of claim 21, wherein the compound is Compound (II-A) of claim 2 and the method comprises contacting hexahydrofarnesoic acid with ascorbic acid, 4-dimethylaminopyridine, N-methyl-2-pyrrolidone, and a coupling agent.

23. The method of claim 22, wherein the coupling agent is selected from N-(3-dimethylaminopropyl)-N′-ethylcarbodiimide hydrochloride, N,N′-diisopropylcarbodiimide, and N,N′-dicyclohexylcarbodiimide.

24. The method of claim 22 or 23, wherein the coupling agent is N-(3-dimethylaminopropyl)-N′-ethylcarbodiimide hydrochloride.

25. The method of claim 21, wherein the compound is Compound (II-B) of claim 3 and the method comprises contacting hexahydrofarnesoic acid with ascorbic acid, 4-dimethylaminopyridine, N-methyl-2-pyrrolidone, dichloromethane, and a coupling agent.

26. The method of claim 25, wherein the coupling agent is selected from N-(3-dimethylaminopropyl)-N′-ethylcarbodiimide hydrochloride, N,N′-diisopropylcarbodiimide, and N,N′-dicyclohexylcarbodiimide.

27. The method of claim 25 or 26, wherein the coupling agent is N-(3-dimethylaminopropyl)-N′-ethylcarbodiimide hydrochloride.

28. The method of claim 21, wherein the compound is Compound (II-C) of claim 4 and the method comprises contacting hexahydrofarnesoic acid with ascorbic acid, 4-dimethylaminopyridine, N-methyl-2-pyrrolidone, ethyl acetate, and a coupling agent.

29. The method of claim 28, wherein the coupling agent is selected from N-(3-dimethylaminopropyl)-N′-ethylcarbodiimide hydrochloride, N,N′-diisopropylcarbodiimide, N,N′-dicyclohexylcarbodiimide, and HATU (hexafluorophosphate azabenzotriazole tetramethyl uronium.

30. The method of claim 28 or 29, wherein the coupling agent is N-(3-dimethylaminopropyl)-N′-ethylcarbodiimide hydrochloride.

31. A method of producing a compound of any one of claims I-4, comprising:
converting hexahydrofarnesoic acid: 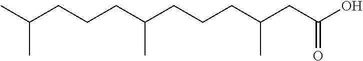 [SMILES: CC(C)CCCC(C)CCCC(C)CC(=O)O]
to hexahydrofarnesyl chloride: 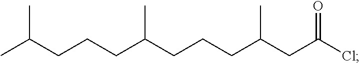 [SMILES: CC(C)CCCC(C)CCCC(C)CC(=O)Cl]
isolating the hexahydrofarnesyl chloride; and
contacting the hexahydrofarnesyl chloride with ascorbic acid and an organic base.

32. The method of claim 31, wherein the compound is Compound (II-A) of claim 2 and the method comprises:
contacting hexahydrofarnesoic acid with dichloromethane, dimethylformamide, and thionyl chloride to form a reaction mixture that comprises hexahydrofarnesyl chloride;
extracting the hexahydrofarnesyl chloride from the reaction mixture; and
contacting the hexahydrofarnesyl chloride with ascorbic acid, pyridine, DMAP, and dichloromethane.

33. The method of claim 31 wherein the compound is Compound (II-C) of claim 4 and the method comprises:
contacting hexahydrofarnesoic acid with dichloromethane, dimethylformamide, and thionyl chloride to form a reaction mixture that comprises hexahydrofarnesyl chloride;
extracting the hexahydrofarnesyl chloride from the reaction mixture; and
contacting the hexahydrofarnesyl chloride with ascorbic acid, pyridine, 4-dimethylaminopyridine, and dichloromethane.

34. The method of any one of claims 6-34, further comprising producing the hexahydrofarnesoic acid via a process that comprises:
contacting hexahydrofarnesol (HHF): 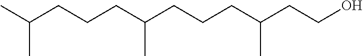 [SMILES: CC(C)CCCC(C)CCCC(C)CCO]
with NaH₂PO₄, TEMPO, and bleach in the absence of solvent; and
adding NaClO₂ over a period between about 4.5 and 6.5 hours.

35. A method of producing hexahydrofarnesoic acid (HHFA): 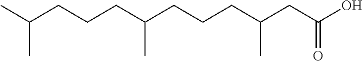 [SMILES: CC(C)CCCC(C)CCCC(C)CC(=O)O]
or a salt and/or stereoisomer thereof comprising:
contacting hexahydrofarnesol (HHF): 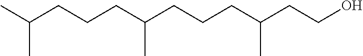 [SMILES: CC(C)CCCC(C)CCCC(C)CCO]
with NaH₂PO₄, TEMPO, and bleach in the absence of solvent; and
adding NaClO₂ over a period between about 4.5 and 6.5 hours.

36. The method of claim 34 or 35, wherein the contact of HHF with NaH₂PO₄ TEMPO, and bleach is conducted at a temperature between −5° C. to about 10° C.

37. The method of any one of claims 34-36, wherein the contact of HHF with NaH₂PO₄ TEMPO, and bleach is conducted at a temperature between about 0° C. to about 5° C.

38. The method of any one of claims 34-37, wherein the TEMPO is an amount that is between about 0.3 and 0.7 equivalence of the HHF.

39. The method of any one of claims 34-38, wherein the concentration of the NaH₂PO₄ is between about 2.5 M and 3.5 M.

40. The method of any one of claims 34-39, wherein the NaH₂PO₄ is an amount that is between about 0.2 and 0.6 equivalence of the HHF.

41. The method of any one of claims 34-40, wherein the concentration of the bleach is between about 10% v/v and 15% v/v.

42. The method of any one of claims 34-41, wherein the bleach is an amount that is between about 0.05 and 0.2 equivalence of the HHF.

43. The method of any one of claims 34-42, wherein the concentration of the NaClO₂ is between about 3 M and about 6 M.

44. The method of any one of claims 34-43, wherein the NaClO₂ is added over a period of about 5 hours.

45. The method of any one of claims 34-43, wherein the NaClO₂ is added over a period between about 4.5 and 6.5 hours at a rate that increases from about 0.5 mL/min and about 1.5 mL/min over the course of addition.

46. The method of any one of claims 34-45, wherein the purity of HHFA is greater than 90%.

47. The method of any one of claims 34-46, wherein the purity of HHFA is greater than 92%.

48. The method of any one of claims 34-47, wherein the purity of HHFA is greater than 95%.

49. The method of any one of claims 34-48, further comprising the bio-fermentation of beta-farnesene from sugarcane.

50. A personal care product comprising a compound selected from

| | |
|---|---|
| Compound (I-A) | 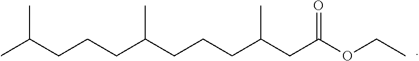 [SMILES: CCOC(=O)CC(C)CCCC(C)CCCC(C)C] Ethyl-hexahydrofarnesoate; |
| Compound (I-B) | 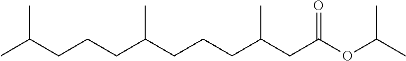 [SMILES: CC(C)CCCC(C)CCCC(C)CC(=O)OC(C)C] Isopropyl-hexahydrofarnesoate; |
| Compound (I-C) | 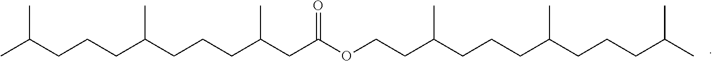 [SMILES: CC(C)CCCC(C)CCCC(C)CCOC(=O)CC(C)CCCC(C)CCCC(C)C] Hexahydrofarnesyl-hexahydrofarnesoate; |
| Compound (II- A) | 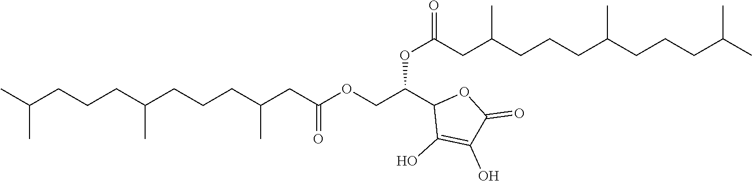 [SMILES: CC(C)CCCC(C)CCCC(C)CC(=O)OC[C@H](OC(=O)CC(C)CCCC(C)CCCC(C)C)C1OC(=O)C(O)=C1O] Dihexahydrofarnesyl ascorbate; |
| Compound (II- B) | 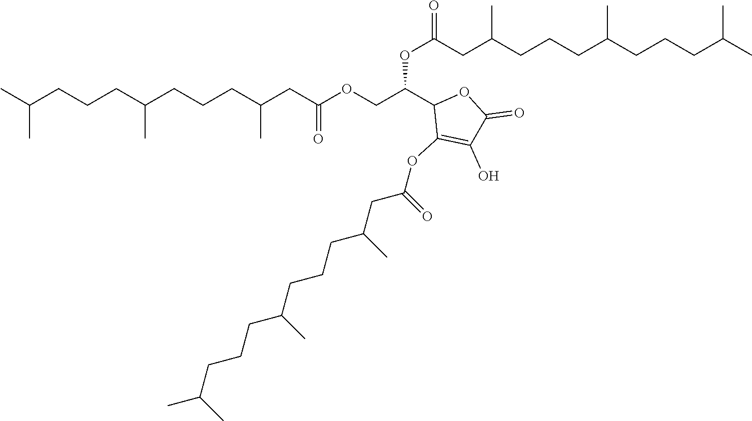 [SMILES: CC(C)CCCC(C)CCCC(C)CC(=O)OC[C@H](OC(=O)CC(C)CCCC(C)CCCC(C)C)C1OC(=O)C(O)=C1OC(=O)CC(C)CCCC(C)CCCC(C)C] Trihexahydrofarnesyl ascorbate; |
| Compound (II- C) | 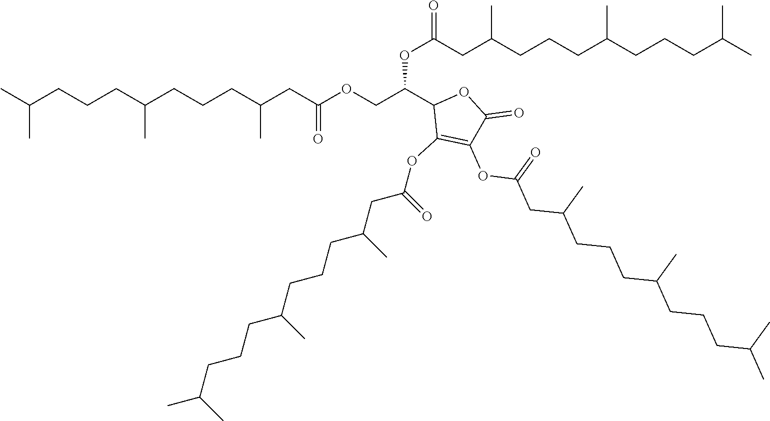 [SMILES: CC(C)CCCC(C)CCCC(C)CC(=O)OC[C@H](OC(=O)CC(C)CCCC(C)CCCC(C)C)C1OC(=O)C(OC(=O)CC(C)CCCC(C)CCCC(C)C)=C1OC(=O)CC(C)CCCC(C)CCCC(C)C] Tetrahexahydrofarnesyl ascorbate; |

or a combination thereof.

51. The personal care product of claim 50, comprising ethyl-hexahydrofarnesoate (Compound (I-A)).

52. The personal care product of claim 50, comprising isopropyl-hexahydrofarnesoate (Compound (I-B)).

53. The personal care product of claim 50, comprising hexahydrofarmesyl-hexahydrofamnesoate (Compound (I-C)).

54. The personal care product of claim 50, comprising dihexahydrofarnesyl ascorbate (Compound (II-A)).

55. The personal care product of claim 50, comprising trihexahydrofarnesyl ascorbate (Compound (II-B)).

56. The personal care product of claim 50, comprising tetrahexahydrofarnesyl ascorbate (Compound (II-C)).

57. The personal care product of any one of claims 50 to 56, further comprising one or more additional components selected from an emollient, a sensory modifier, an organic solvent, a co-emulsifier, a thickener, a moisturizing agent, an anti-inflammatory agent, a preservative, an anti-aging active, an anti-oxidant, water, and a fragrance.

58. The personal care product of claim 57, wherein the fragrance comprises patchoulol.

59. The personal care product of claim 57, wherein the moisturizing agent comprises ectoine.

60. The personal care product of claim 57, wherein the thickener comprises carboxymethyl holocellulose.

61. The personal care product of claim 57, wherein the emollient is selected from hexahydrofarnesyl-hexahydrofarnesoate (Compound (I-C)), ethyl-hexahydrofarnesoate (Compound (I-A)), and isopropyl-hexahydrofamesoate (Compound (I-B)).

62. The personal care product of claim 57, wherein the anti-inflammatory agent comprises a cannabinoid.

63. The personal care product of claim 62, wherein the cannabinoid is cannabigerol.

64. The personal care product of claim 57, wherein the emulsifier is selected from hexahydro-farnesoic acid and hexahydro-farnesol.

65. The personal care product of claim 57, wherein the anti-aging active is selected from dihexahydrofarnesyl ascorbate (Compound (II-A)), trihexahydrofarnesyl ascorbate (Compound (II-B)), and tetrahexahydrofarnesyl ascorbate (Compound (II-C)).

66. The personal care product of any one of claims 50 to 65, wherein all of the components are derived from sugar cane.

67. The personal care product of claim 66, wherein one or more components are derived from bio-farnesene wherein the bio-farnesene is produced by fermentation of sugar cane.

68. The personal care product of any one of claims 50 to 67, further comprising a hexahydrofamesene dimer (HDD) selected from the group consisting of: [chemical structure]
